SEQUENCE LISTING (10 sequences)

SEQ ID NO: 1 - RNA, 21 nt, Human
cauaaaguagaaagcacuacu

SEQ ID NO: 2 - RNA, 87 nt, Human
gacagugcagucacccauaaaguagaaagcacuacuaacagcacuggaggguguaguguuuccuacuuuauggaugaguguacugug

SEQ ID NO: 3 - RNA, 23 nt, Human
uguaguguuuccuacuuuaugga

SEQ ID NO: 4 - RNA, 24 nt, Human
uuuaaugaaucacuaagcuuuauu

SEQ ID NO: 5 - RNA, 24 nt, Bos taurus (cow)
uuuaaugaaucacuaagcuuuauu

SEQ ID NO: 6 - RNA, 24 nt, Sus scrofa (pig)
uuuaaugaauuacuaagcuuuauu

SEQ ID NO: 7 - RNA, 24 nt, Felis Catus (cat)
uuuaaugaaucaccgagcuuuauu

SEQ ID NO: 8 - RNA, 24 nt, Rattus norvegicus (rat)
uuuaaugaaucacuacacuuuauu

SEQ ID NO: 9 - RNA, 24 nt, Pan troglodytes (chimp)
uuuaaugaaucacuaagcuuuauu

SEQ ID NO: 10 - RNA, 24 nt, Mus musculus (mouse)
uuuaaugaaucacuacacuuuauu

(19) United States
(12) Patent Application Publication
(10) Pub. No.: US 2022/0054548 A1
(43) Pub. Date: Feb. 24, 2022

(54) MIRNA FOR USE IN THERAPY

(71) Applicant: King's College London, London (GB)

(72) Inventor: Graham Michael Lord, London (GB)

(73) Assignee: King's College London, London (GB)

(21) Appl. No.: 17/415,262

(22) PCT Filed: Dec. 20, 2019
(86) PCT No.: PCT/EP2019/086739

§ 371 Date: Jun. 17, 2021

(30) Foreign Application Priority Data
Dec. 21, 2018 (GB) 1321147.4

(51) Int. Cl. A61K 35/17; C12N 5/0783; C12N 15/113; A61P 37/06

(52) U.S. Cl. CPC A61K 35/17; C12N 5/0637; C12N 2510/00; A61P 37/06; C12N 2310/141; C12N 15/1137

(57) ABSTRACT

The present invention relates to a modified T regulatory cell (Treg) in which the level of microRNA miR-142-5p (CAUAAAGUAGAAAGCACUACU) or a variant thereof is increased or decreased. Therapeutic uses of said modified Tregs are also provided, in particular in the treatment of autoimmune diseases and cancer. Populations of said Tregs and methods of preparing such Tregs are also provided.

### FIELD OF THE INVENTION

The present invention relates to T regulatory (Treg) cells which have been modified to increase or reduce expression of a particular miRNA, miR-142-5p, and therapeutic methods using such Tregs, for example in the treatment of autoimmune diseases and cancer. More particularly, the invention relates to therapeutic methods in the form of T cell therapies, for example adoptive T cell therapies, or other therapeutic methods which comprise the administration of miRNAs.

### BACKGROUND OF THE INVENTION

MicroRNAs (miRNAs) are a class of small (˜21-25 nucleotides), highly-conserved, endogenous non-coding RNAs that regulate gene expression post-transcriptionally. This function is mediated through binding of the miRNA-containing RNA-induced silencing complex (miRISC) to complementary sequences in the 3′ untranslated region (3′UTR) of target messenger RNAs (mRNAs), ultimately causing mRNA degradation or translational inhibition (1,2). Through these regulatory actions, miRNAs have been shown to be critical for normal innate and adaptive immune processes (3), with aberrant expression implicated in multiple autoimmune diseases and malignancies (4,5). Potential susceptibility to pharmacological manipulation with modified antisense oligonucleotides also makes them attractive therapeutic targets (6).

Regulatory T cells (T_{REGS}) are primarily CD4 positive and play an essential role in maintenance of peripheral tolerance and avoidance of autoimmunity through the direct suppression of self-reactive T effector cells (T_{EFFS}). The T_{REG} lineage is defined by expression of the transcription factor, Forkhead box P3 (FOXP3) also known as ‘Scurfin’, whose gene locus is located on the X chromosome (7). FOXP3 is exclusively expressed by T_{REGS} and is critical for T_{REG} lineage commitment. The importance of T_{REGS} for maintenance of peripheral tolerance was highlighted by studies of the Scurfy mouse, which lacks functional T_{REGS} due to a missense mutation in the murine Foxp3 gene. These mice develop a severe lymphoproliferative disease with generalised multi-organ inflammation, leading to death by 24 days of age (8,9). A similar outcome is observed in human patients suffering from immunodysregulation, polyendocrinopathy, enteropathy, X-linked (IPEX) syndrome. IPEX syndrome is also caused by mutations in the FOXP3 gene and characterized by defective T_{REGS}, multi-system inflammation and autoimmunity, with death usually by 2 years of age unless successfully treated (10,11). Importantly, mice with a T_{REG}-specific deletion of either Dicer (12-14) or Drosha (15), the two ribonuclease III (RNase III) enzymes necessary for the production and processing of mature miRNA species, also develop a spontaneous, lethal autoimmune disease virtually indistinguishable from that seen in Scurfy mice (7) demonstrating that miRNAs are critical for establishment of T_{REG}-mediated peripheral tolerance. However, the set of miRNAs responsible for this functional deficiency has yet to be fully defined.

MicroRNA-142 (miR-142) is one of a handful of hematopoietic-specific miRNAs (16) and exists as two mature isoforms—miR-142-3p and miR-142-5p, generated by ribonuclease processing of the sense and antisense strands of the intact double-stranded miR-142 duplex. Of the two mature species, miR-142-5p is the predominant form expressed in thymically-derived T_{REGS} (17). Importantly, the mature sequence of miR-142 is evolutionarily conserved between murine and human species, making it an attractive target for translation from murine studies to human clinical use (18). miR-142-5p expression is down-regulated in CD4⁺ T cells from patients with the multisystem autoimmune disease Systemic Lupus Erythematosis (SLE) compared to healthy controls and over-expressed in an animal model of multiple sclerosis, suggesting that miR-142 plays a role in autoimmune disease (19, 20).

### SUMMARY OF THE INVENTION

The present inventors have shown that miR-142-5p directly targets phosphodiesterase-3b (Pde3b), mRNA, limiting PDE3B protein levels in T_{REGS}. PDE3B (cyclic nucleotide phosphodiesterase 3B, cyclic guanosine monophosphate-inhibited) hydrolyses its substrates 3′-5′-cyclic adenosine monophosphate (cAMP) and 3′-5′-cyclic guanine monophosphate to adenosine monophosphate (AMP) and guanine monophosphate (GMP), respectively, thus accelerating intracellular cAMP and cGMP turnover (21). The suppressive function of T_{REGS} is critically dependent on the intracellular concentration of cAMP (22), with T_{REGS} maintaining high levels of intracellular cAMP and T_{EFFS} requiring low cAMP levels to undergo activation (23). Collectively, the findings of the present inventors reveal a critical role for miR-142-5p in the regulation of intracellular cAMP concentration via modulation of Pde3b expression in T_{REGS} and, therefore, places miR-142-5p in the center of the molecular circuitry that regulates T_{REG} suppressive function.

Thus, the present inventors have shown that miR-142-5p has a role in Treg function, in particular a role in the ability of Tregs to suppress Teff proliferation. Indeed, the present inventors have shown that miR-142-5p appears to be essential for the ability of Tregs to suppress Teff proliferation.

The present inventors have further shown that miR-142-5p directly targets PDE3B mRNA, specifically the 3′UTR of the PDE3B mRNA, in order to reduce PDE3B expression (represses PDE3B). Advantageously, this reduction in expression (repression) is specific to (or selective for) PDE3B as opposed to PDE 3A. This is in direct contrast to other pharmaceutical agents currently being used, which inhibit both PDE3B and PDE3A (e.g. cilostazol).

Thus, the inventors have shown that Tregs in which the miR-142-5p has been deleted lose the ability to suppress Teff proliferation. This is believed to be due to the Tregs having increased PDE3B expression, which in turn results in reduced intracellular cAMP levels. Indeed, mice with Tregs in which miR-142-5p has been deleted, show symptoms of autoimmune disease similar to the Scurfy mouse (in which FOXP3 has been deleted). Surprisingly, the autoimmune disease in these miR-142-5p defective mice can be significantly alleviated or treated by inhibition of PDE3B either through chemical means (e.g. using small molecule inhibitors) or by ablating the PDE3B gene in the mouse model.

Thus, following these findings, it can be seen that inhibiting or reducing PDE3B levels (e.g. protein or mRNA levels), or activity (e.g. using pharmaceutical agents such as those discussed elsewhere herein) can be used therapeutically in order to treat autoimmune diseases. Thus, at its broadest the present invention provides an agent which inhibits or reduces PDE3B levels (e.g. protein or mRNA levels), or activity, for use in the treatment of autoimmune diseases (or any other diseases in which PDE3B level or activity is increased or upregulated, or a disease associated with or characterised by aberrant or excessive PDE3B).

Thus, the present invention further provides a method of treating autoimmune disease in a subject, said method comprising the step of administrating an effective amount of an agent which inhibits or reduces PDE3B levels (e.g. protein or mRNA levels) or activity, to said subject. Such methods are also appropriate for the treatment of any other disease in which PDE3B level or activity is increased or upregulated or a disease associated with or characterised by aberrant or excessive PDE3B.

The present invention further provides the use of an agent which inhibits or reduces PDE3B levels (e.g. protein or mRNA levels) or activity, in the manufacture of a medicament, or composition, for the treatment of autoimmune disease. Such uses are also appropriate for the treatment of any other disease in which PDE3B level or activity is increased or upregulated or a disease associated with or characterised by aberrant or excessive PDE3B.

Alternative and preferred embodiments and features of the invention as described elsewhere herein apply equally to these methods of treatment and uses of the invention.

The demonstration that miR-142-5p has a direct effect on inhibiting or reducing PDE3B levels means that miR-142-5p can also be used therapeutically in order to treat autoimmune diseases. Thus, in a further aspect, the present invention provides miR-142-5p, for example miR-142-5p (CAUAAAGUAGAAAGCACUACU, SEQ ID NO:1) or a variant thereof, for use in the treatment of autoimmune diseases (or any other diseases in which PDE3B level or activity is increased or upregulated or a disease associated with or characterised by aberrant or excessive PDE3B).

Thus, the present invention further provides a method of treating autoimmune disease in a subject, said method comprising the step of administrating an effective amount of miR-142-5p, for example miR-142-5p (CAUAAAGUAGAAAGCACUACU, SEQ ID NO:1) or a variant thereof, to said subject. Such methods are also appropriate for the treatment of any other disease in which PDE3B level or activity is increased or upregulated or a disease associated with or characterised by aberrant or excessive PDE3B.

The present invention further provides the use of miR-142-5p, for example miR-142-5p (CAUAAAGUAGAAAGCACUACU, SEQ ID NO:1) or a variant thereof, in the manufacture of a medicament, or composition, for the treatment of autoimmune disease. Such uses are also appropriate for the treatment of any other disease in which PDE3B level or activity is increased or upregulated or a disease associated with or characterised by aberrant or excessive PDE3B.

Alternative and preferred embodiments and features of the invention as described elsewhere herein apply equally to these methods of treatment and uses of the invention.

More specifically, the demonstration that miR-142-5p has an effect on the function of Tregs to suppress Teffs, for example by inhibiting or reducing PDE3B levels, means that Tregs which are modified to increase or reduce expression of miR-142-5p, for example miR-142-5p (CAUAAAGUAGAAAGCACUACU, SEQ ID NO:1) or a variant thereof, can be used therapeutically in order to treat autoimmune diseases (where miR-142-5p levels are increased) or to treat cancer (where miR-142-5p levels are decreased). Such modified Tregs and their therapeutic uses thus form further aspects of the invention.

Thus, in a preferred aspect, the present invention provides a regulatory T cell (Treg) in which the level of miR-142-5p (CAUAAAGUAGAAAGCACUACU, SEQ ID NO:1) or a variant thereof is increased.

The present invention further provides a regulatory T cell (Treg) in which the level of miR-142-5p (CAUAAAGUAGAAAGCACUACU, SEQ ID NO:1) or a variant thereof is increased, for use in therapy, in particular for use in the treatment of autoimmune disease.

Thus, the present invention further provides a method of treating autoimmune disease in a subject, said method comprising the step of administrating an effective amount of a regulatory T cell (Treg) in which the level of miR-142-5p (CAUAAAGUAGAAAGCACUACU, SEQ ID NO:1) or a variant thereof is increased, to said subject.

The present invention further provides the use of a regulatory T cell (Treg) in which the level of miR-142-5p (CAUAAAGUAGAAAGCACUACU, SEQ ID NO:1) or a variant thereof is increased, in the manufacture of a medicament, or composition, for the treatment of autoimmune disease.

Alternative and preferred embodiments and features of the invention as described elsewhere herein apply equally to these methods of treatment and uses of the invention.

A yet further aspect of the invention provides a T regulatory (Treg) cell in which the level of miR-142-5p (CAUAAAGUAGAAAGCACUACU, SEQ ID NO:1) or a variant thereof is reduced.

The present invention further provides a regulatory T cell (Treg) in which the level of miR-142-5p (CAUAAAGUAGAAAGCACUACU, SEQ ID NO:1) or a variant thereof is reduced, for use in therapy, in particular for use in the treatment of cancer.

Thus, the present invention further provides a method of treating cancer in a subject, said method comprising the step of administrating an effective amount of a regulatory T cell (Treg) in which the level of miR-142-5p (CAUAAAGUAGAAAGCACUACU, SEQ ID NO:1) or a variant thereof is reduced, to said subject.

The present invention further provides the use of a regulatory T cell (Treg) in which the level of miR-142-5p (CAUAAAGUAGAAAGCACUACU, SEQ ID NO:1) or a variant thereof is reduced, in the manufacture of a medicament, or composition, for the treatment of cancer.

Alternative and preferred embodiments and features of the invention as described elsewhere herein apply equally to these methods of treatment and uses of the invention.

In all the therapeutic methods and uses of the invention, the therapeutic agents, e.g. Tregs or miR-142-5p molecules, or other agents, are preferably administered in pharmaceutically or physiologically effective amounts, to a subject/patient in need thereof.

In all of the embodiments herein, the Tregs are preferably human Tregs. In all of the embodiments herein, the subjects/patients are preferably human subjects.

### BRIEF DESCRIPTION OF THE DRAWINGS

FIG. 1: T_{REG}^{Δ142} mouse: validation data. (A) ChIP-seq binding profiles (reads/million, input subtracted) for FOXP3 and H3K4me3 and mRNA-seq (reads/million) around miR142 in T_{REGS}. Genes and super-enhancers are shown below and a scale bar above. T_{REGS} are defined as CD4⁺CD25⁺FOXP3⁺. (B) Flow cytometric gating on YFP (sorted and fixed live CD4+ cells) and concomitant/subsequent FOXP3 staining. (C) miR-142-5p expression in naïve CD4⁺ T cells and YFP⁺ T_{REGS} in WT and T_{REG}^{Δ142} by RT-qPCR. (n≥3; *p<0.05; **p<0.001, two-tailed Student's t-test). (D) Total CD3 counts in spleen (n=4 per group) and peripheral lymph node (n=7 per group) (non-significant, two-tailed Student's t-test). (E) Total CD4 counts in spleen and peripheral lymphnode in the absence of disease (<10 weeks; n=5; non-significant, two-tailed Student's t-test) and presence of disease (>10 weeks; n=3; non-significant, two-tailed Student's t-test).

FIG. 2: MiR-142-deficient T_{REGS} develop normally, but fail to suppress effector T cell responses in vitro. (A) Number and proportion (%) of FOXP3⁺ (CD4⁺YFP⁺) cells at successive stages of thymus development (n=4 per group). (B) Number of FOXP3⁺ cells in spleen (left) and peripheral lymph node (pLN) (right) samples (n=8). (C) Flow cytometry histograms of peripheral T_{REGS} (CD4⁺CD25⁺FOXP3⁺) stained for surface and intracellular T_{REG} markers (n≥4 per group). (D) Flow cytometry and cytokine secretion profiles of CD3⁺CD4⁺ and CD3⁺CD8⁺ T cells (*p<0.05, **p<0.01, two-tailed Student's t-test, n≥4 per group). (E) Flow cytometry and ICC data for CD25 surface expression and IL-2 production in T_{REG}^{Δ142} (CD4⁺YFP⁺) vs. WT T_{REGS} (CD4⁺CD25⁺FOXP3⁺) (**p<0.01, two-tailed Student's t-test, n=6 per group). (F) Co-culture suppression assays; p-value represents comparison of WT T_{REGS}:WT T_{EFFS} and T_{REG}^{Δ142}:WT T_{EFFS} (data combined from 3 independent experiments). CTV=Cell Trace Violet. (*p<0.05, two-tailed student's t-test, n>3 per group).

FIG. 3: T_{REG}-specific deficiency of mir-142 causes a multi-system lethal autoimmune syndrome due to a failure of peripheral tolerance. (A) Weight charts demonstrating weight loss from 7 weeks of age in T_{REG}^{Δ142} males and females (***p<0.001, two-tailed Student's t-test; n>10 per group). WT and T_{REG}^{Δ142} data are also shown in FIG. 4A (B) Survival of T_{REG}^{Δ142} and WT littermate control mice (***p<0.001 two-tailed Student's t-test, data combined from 2 independent experiments; n>10). (C) “Scurfy”-type phenotype seen in T_{REG}^{Δ142} mice (16 week old female mouse shown), gross splenomegaly and lymphadenopathy seen in T_{REG}^{Δ142} mice. (D) H&E staining of FFPE sections from ear skin (×20 magnification), liver (×10 magnification) and lung (×10 magnification); WT and T_{REG}^{Δ142} skin, lung and liver histology are also shown in FIG. 6E.

FIG. 4: The cell intrinsic T_{REG} suppressive defect is directly attributable to cell-specific loss of miR-142 expression. (A) Weight charts of WT, T_{REG}^{Δ142} and Foxp3^{YFP-Cre}×Mir142^{fl/+} mice (male and female; n>9). WT and T_{REG}^{Δ142} data are also shown in FIG. 3A (B) Co-culture suppression assays (data combined from 3 independent experiments); p-values represent comparison between WT, T_{REG}^{Δ142}, Foxp3^{YFP-Cre/WT}×Mir142^{fl/fl} and Foxp3^{YFP-Cre}×Mir142^{fl/+} (**p<0.01, ***p<0.001 one way ANOVA, n>4 per group); no significant difference noted between WT and Foxp3^{YFP-Cre}×miR-142^{fl/+}. (C) H&E staining of FFPE sections from ear skin, liver and lung from Foxp3^{YFP-Cre/WT}×Mir142^{fl/fl} and Foxp3^{YFP-Cre}×Mir142^{fl/+} mice (×10 magnification). (D) Comparison of spleen weights and cell counts from WT, T_{REG}^{Δ142}, Foxp3^{YFP-Cre/WT}×Mir142^{fl/fl} (female) and Foxp3^{YFP-Cre}×Mir142^{fl/+} (male and female) mice (*p<0.05, **p<0.01; one-way ANOVA, n>6). (E) YFP+ T_{REGS} as a percentage of the total T_{REG} pool (CD4⁺CD25⁺FoxP3⁺) from WT, T_{REG}^{Δ142}, Foxp3^{YFP-Cre/WT}×Mir142^{fl/fl} (female) and Foxp3^{YFP-Cre}×Mir142^{fl/+} (male and female) mice (n=4 per group; ***p<0.001; one-way ANOVA). (F) Weights of WT, T_{REG}^{Δ142} and Foxp3^{YFP-Cre/WT}×Mir142^{fl/fl} (female) (n>6 per group; one-way ANOVA). (G) Cytokine secretion profiles of CD3⁺CD4⁺ and CD3⁺CD8⁺ T cells from WT, T_{REG}^{Δ142}, Foxp3^{YFP-Cre/WT}×Mir142^{fl/fl} (female) and Foxp3^{YFP-Cre}×Mir142^{fl/+} mice (male and female) (n≥3, **p<0.01. ***p<0.001: one-way ANOVA).

FIG. 5: Identification of candidate miR-142 target genes in T_{REGS} (A) Intersection of genes harboring miR-142-5p binding sites in their 3′ UTRs, as predicted by DIANA microT algorithm and by AGO2 HITS-CLIP in activated CD4⁺T cells (29) (blue, (1)), with genes down-regulated (≥2-fold, p<0.05) in T_{REGS} vs T_{EFFS} cells (30) (green, (3)) and genes up-regulated in miR-142-deficient versus WT T_{REGS} (≥2-fold, p<0.05) (yellow, (2)). (B) Direct targeting of Pde3b mRNA 3′ UTR by miR-142. Expression of an NGFR reporter gene that has either the wild type (WT) or mutated (Mut) Pde3b 3′UTR miR-142-5p binding site inserted in the presence of control or miR-142 expression vector in HEK293T cells. (Data are representative of two independent experiments. Bars indicate mean and SD, n=8; ***p<0.001, n.s.=not significant, Student's t-test). (C) Pde3b expression in T_{REG}^{Δ142}, Pde3b^{-/-} and WT T_{REGS} by RT-qPCR. (n=6; ***p<0.001, two-tailed Student's t-test. Data from 2 independent experiments). (D) PDE3B expression in WT and T_{REG}^{Δ142} T_{REGS} by Western blot (relative density; n≥3; **p<0.01, two-tailed Student's t-test; lanes were run on the same gel but were noncontiguous). (E) cAMP ELISA data from T_{REG} cell lysates (n=4; **p<0.001, two-tailed Student's t-test). (F) Co-culture suppression assay data comparing T_{REG} suppressive function following in vitro cilostamide treatment of T_{REGS} from WT and T_{REG}^{Δ142} mice; p-value signifies comparison between T_{REG}^{Δ142}+cilostamide and T_{REG}^{Δ142}+control; no significant difference between T_{REG}^{Δ142}+cilostamide and WT samples (**p<0.01, one-way ANOVA, n≥9 per group, combined from 3 independent experiments). (G) Co-culture suppression assay data comparing T_{REG} suppressive function in WT and Pde3b^{-/-} mice (**p<0.01, one-way ANOVA, n≥6 per group, combined from 2 independent experiments).

FIG. 6: Pharmacological inhibition of PDE3B or genetic deletion of Pde3b reverses the lethality and phenotype of the autoimmune syndrome induced by the T_{REG}-specific loss of miR-142 (A) Weight (left) and survival (right) of T_{REG}^{Δ142} and WT littermate control mice after 8-weeks of treatment with 6.4 mg/kg intra-peritoneal cilostamide or control (n≥3 for WT mice and n≥6 for T_{REG}^{Δ142} mice). Loss of more than 15% of body weight was the predefined mortality endpoint). (B) Co-culture T_{REG} suppression assay after 4 weeks of cilostamide treatment (*p<0.05, **p<0.01, ***p<0.001, one-way ANOVA, data combined from 3 independent experiments). (C) Weight (left) and survival (right) of Pde3b^{-/-}×T_{REG}^{Δ142} (3); T_{REG}^{Δ142} (2); Pde3b^{+/-}×T_{REG}^{Δ142} (4) and WT mice (1) (n≥5 for Pde3b^{-/-}×T_{REG}^{Δ142}; n≥3 for Pde3b^{+/-}×T_{REG}^{Δ142}; n≥7 for T_{REG}^{Δ142} and n≥5 for WT mice). (D) Co-culture T_{REG} suppression assay comparing germline deletion of Pde3b (Pde3b^{-/-}×T_{REG}^{Δ142}) with T_{REG}^{Δ142} and WT littermate control mice; p-values signify comparison between Pde3b^{-/-}×T_{REG}^{Δ142} and T_{REG}^{Δ142} (*p<0.05, **p<0.01, one-way ANOVA, data combined from 2 independent experiments, n≥6). (E) H&E staining of FFPE sections from ear skin (×20 magnification), liver (×10 magnification) and lung (×10 magnification) (left) with histological scoring as described in materials and methods (right) (*p<0.05, Student's t-test, n=5 Pde3b^{-/-}×T_{REG}^{Δ142} (3), T_{REG}^{Δ142} (2) and WT (1); n=3 Pde3b^{+/-}×T_{REG}^{Δ142} (4)); WT and T_{REG}^{Δ142} skin, lung and liver histology are also shown in FIG. 3D.

In the attached Figures Δ142 is sometimes denoted as □142.

### DETAILED DESCRIPTION OF THE INVENTION AND PREFERRED EMBODIMENTS THEREOF

The present invention provides a regulatory T cell (Treg) in which the level of miR-142-5p (CAUAAAGUAGAAAGCACUACU, SEQ ID NO:1) or a variant thereof is increased.

microRNAs (miRNAs) are small, highly conserved, endogenous, non-coding RNAs with important roles in gene regulation. For example, miRNAs can inhibit gene expression in a post-transcriptional manner by affecting both the stability and translation of mRNAs. MicroRNAs generally have a suppressive action on their target genes. The final miRNA products which inhibit gene expression are small single-stranded RNA molecules, approximately 18 to 25 nucleotides long, e.g. 21 to 25 or 20 to 24 nucleotides long.

miRNA gene transcription is carried out by RNA polymerase II in the nucleus to give primary miRNA (pri-miRNA), which is a 5′ capped, 3′ polyadenylated RNA with double stranded stem-loop structure. The pri-miRNA is then cleaved by a microprocessor complex (comprising Drosha, which is a ribonuclease III enzyme, and microprocessor complex subunit DCGR8) to form precursor miRNA (pre-miRNA), which is a duplex that contains 70 to 100 nucleotides with interspersed mismatches and adopts a stem-loop structure. The pre-miRNA is subsequently transported by Exportin 5 from the nucleus to the cytoplasm, where it is further processed by Dicer (a ribonuclease III enzyme) into a miRNA duplex (also called a mature miRNA duplex) of 18 to 25 nucleotides wherein the 3′ end corresponding to the 3′ end of the pre-miRNA generally has a 2 nucleotide overhang. The miRNA duplex then associates with, or is incorporated into, a RNA-induced silencing complex (RISC), to form a complex called miRISC. The miRNA duplex is then unwound, releasing and discarding one of the strands, the passenger strand, which is not used for the inhibition of gene expression. The other remaining strand of the duplex, i.e. the guide strand of the mature duplex miRNA, guides the miRISC to the target mRNAs. The miRNA (the single-stranded guide strand) binds to the target mRNAs through partial complementary base pairing with the consequence that the target gene silencing occurs via translational repression, mRNA degradation, and/or mRNA cleavage.

For example, micro-RNAs often have a target region in the 3′ UTR of an mRNA transcript. Thus, miRNA only needs to be partially complementary to its target mRNA in order to affect gene expression. The complementary pairing between mRNA and the mature miRNA typically occurs at the 3′ UTR of the mRNA and involves the seed region (generally nucleotides 2 to 7 from the 5′ end) of the mature single-stranded miRNA. Since miRNA recognition does not require perfect complementary pairing, one miRNA strand can recognise an array of mRNAs, and hence miRNA can have the characteristic of having multiple targets.

Many miRNAs have been identified, although the specific roles and targets of many miRNAs are still rather elusive. The present invention is concerned in particular with miR-142.

The micro-RNA-142 (miR-142) has a pre-miRNA-142 structure which has a signature stem-loop structure, and which encodes two micro-RNA species (miR-142-5p and miR-142-3p). The human coding sequence for the pre-miRNA-142 is shown below (SEQ ID NO:2). The pre-miR-142 encodes both the miR-142-5p and miR-142-3p.

(SEQ ID NO: 2)
GACAGUGCAGUCACCCAUAAAGUAGAAAGCACUACUAACAGCACUGGAGG
G [symbol] [symbol] UGAGUGUACUGUG.

The miR-142-5p sequence is shown underlined and the miR-142-3p sequence is shown in bold italics. The sequence is 87 nucleotides in length, with the miR-142-5p sequence at nucleotides 16-36 and the miR-142-3p sequence at nucleotides 52-74.

The stem loop structure of the human pre-miR-142 is shown below, with the miR-142-5p on the top strand and the miR-142-3p on the bottom strand of this figure.

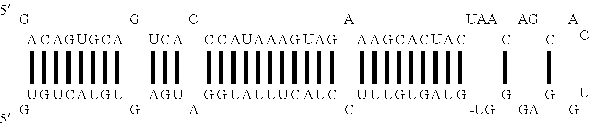

It has been shown that the promoter of miR-142 contains separate binding sites for three transcription factors, PU.1, C/EBPb and Runx1.

Thus, the sequence of miR-142-5p is:

CAUAAAGUAGAAAGCACUACU (SEQ ID NO:1, 21 nucleotides) and
the sequence of miR-142-3p is:
UGUAGUGUUUCCUACUUUAUGGA (SEQ ID NO:3, 23 nucleotides)

Although miR-142 encodes 2 types of miRNA, miR-142-5p and miR-142-3p, the present invention is concerned with miR-142-5p, in particular with modifying the levels, e.g. expression levels, of miR-142-5p. Thus, for the present invention, the miR-142-5p would be in the guide strand of the miRNA duplex and the miR-142-3p would be in the passenger strand.

As discussed elsewhere herein, the miR-142-5p has been shown to directly target and reduce expression (e.g. reduce mRNA or protein levels) of PDE3B. The sequence on PDE3B, which is present in the 3′UTR of PDE3B and with which miR-142-5p interacts is 5′ UUUAAUGAAUCACUAAGCUUUAUU 3′ (SEQ ID NO:4, see Table 2). It can be noted that this human 3′UTR sequence is highly conserved across species (see Table 2), in particular the human and primate (chimp) sequences are identical, and there is a high level of conservation between human and mouse sequences. The seed sequence in miR-142-5p, which is a key component of miR-142-5p in order for the interaction with the 3′UTR of PDE3B to take place is located at nucleotides 2 to 7 of miR-142-5p (AUAAAG) and is also shown in Table 2. It can be seen that although there is not 100% complementarity between the miR-142-5p sequence and the 3′UTR over the whole length of the miR-142-5p, there is 100% complementarity between the seed region and the 3′UTR. It can also be noted that the region of the 3′UTR which interacts with the seed region is fully conserved across mammalian species, for example the species shown in Table 2. Indeed, all of the residues in the 3′ UTR of PDE3B which are complementary to (and are in a position to interact with) corresponding residues in miR-142-5p are fully conserved across mammalian species, for example the species shown in Table 2.

In some embodiments of the present invention, the Tregs have an increased level of miR-142-5p (CAUAAAGUAGAAAGCACUACU, SEQ ID NO:1) or a variant thereof. However, there are multiple ways of achieving such an increased level and any of these may be used. For example, the increased level of miR-142-5p may be achieved by way of increasing one of the native precursor molecules of miR-142-5p (or a variant thereof), e.g. pri-mRNA (which can interact with Drosha), pre-miRNA (which can interact with Dicer) or mature duplex miRNA (which can interact with RISC or AGO (Argonaut) protein, e.g. AGO2). Thus, further aspects of the invention provide Tregs in which the level of a precursor of miR-142-5p (CAUAAAGUAGAAAGCACUACU, SEQ ID NO:1) or a variant thereof is increased, e.g. Tregs in which the level of one or more of pri-mRNA, pre-miRNA, or mature duplex miRNA, or a variant thereof, is increased.

The miR-142-5p functions to block or reduce or inhibit expression of PDE3B by being able to bind to the 3′ UTR of the PDE3B mRNA. Thus, in embodiments of the invention in which the level of miR-142-5p (or a variant thereof) is increased, this is preferably accompanied by the miR-142-5p (or a variant thereof) binding to (or being able to bind to) the 3′UTR of PDE3B and/or reducing the expression of PDE3B (and vice versa for embodiments in which the level of miR-142-5p (or a variant thereof) is decreased). As with other miRNA molecules the inhibition is thought to be via a mixture of translational repression, mRNA degradation (e.g. by deadenylation, decapping or exonuclease action) and mRNA cleavage (typically by endonucleolytic cleavage induced by the AGO protein, e.g. AGO2, which is a protein component of the RNA-induced silencing complex, RISC). Translational repression and mRNA degradation are thought to be the primary mediators of the reduced expression of PDE3B mRNA as levels of both PDE3B protein and PDE3B mRNA are affected. Thus, the term “expression”, “overexpression”, “reduced expression”, “inhibited expression”, etc., and equivalent terms as used herein, for example with reference to PDE3B, can refer to protein expression and/or mRNA expression.

Thus, modified or variant versions (also referred to herein as substantially homologous sequences) of the native sequences of miR-142-5p (or miR-142-5p precursors) may be used providing that these modifications retain the appropriate function of miR-142-5p (or miR-142-5p precursor).

Thus, if a sequence of a pri-miRNA or pre-miRNA for miR-142-5p is involved in obtaining increased levels of miR-142-5p, then the modified or variant sequences preferably need to retain the ability to be exported from the nucleus of the Treg cell, e.g. via Exportin 5 (and preferably to be correctly processed by Drosha in the case of a pri-miRNA molecule). Such molecules also preferably retain the ability to be processed (e.g. correctly processed) by the intracellular cytoplasmic machinery (e.g. proteins such as Dicer), and/or interact with the intracellular proteins responsible for targeting the miRNA (the guide strand of the miRNA) to the target mRNA (e.g. the proteins making up the RISC or the AGO protein, e.g. AGO2). When it comes to the ability to interact with or be loaded into the RISC, the molecule also needs to retain the ability to be loaded so that the miR-142-5p is the guide strand and the miR-142-3p is the passenger strand. Ultimately, the modified or variant sequences also need to be able to bind to the 3′UTR of PDE3B and/or to reduce expression of PDE3B.

Thus, if the invention is being carried out with a form of miR-142-5p that does not require interaction with the intracellular processing proteins such as Dicer, RISC or AGO (e.g. certain duplex miRNA molecules or single stranded RNA molecules which correspond to or mimic a miR-142-5p), then the modified or variant sequences do not need to necessarily retain the ability to be processed by the intracellular machinery (e.g. Dicer), or interact with the intracellular proteins responsible for targeting the miRNA (the guide strand of the miRNA) to the target mRNA (e.g. the proteins making up the RISC or the AGO protein). In this case, the key feature of the modified or variant sequences is that they retain the ability to bind to the 3′UTR of PDE3B and/or to reduce expression of PDE3B.

Thus, if for example a single stranded form of miR-142-5p is being used (e.g. alone or encoded by a miR-142-5p precursor), then the modified or variant sequences should retain or have the ability to bind to the 3′UTR of PDE3B and/or to reduce expression of PDE3B. For example, sequence variants of CAUAAAGUAGAAAGCACUACU (SEQ ID NO:1) can be used providing this ability is retained or present.

Dicer-ready or Dicer substrate miRNA, i.e. miRNA which is subjected to Dicer processing, may have the advantage of being more efficiently loaded into the RISC, thereby potentially improving the subsequent gene silencing mechanism by mimicking the naturally occurring process. Thus, such miRNAs are preferred for use in the present invention.

Appropriate variant or substantially homologous sequences might comprise or consist of a nucleotide sequence with a sequence identity of at least 60%, 65%, 70% or 75%, preferably at least 80%, and even more preferably at least 85%, 90% or 95% sequence identity to the native miR-142-5p sequence disclosed herein. Thus, sequences longer than the native miR-142-5p sequence may be used.

These variant or substantially homologous sequences should retain or have one or more of the various appropriate functional properties as outlined above. Ultimately and preferably the final single stranded miRNA product or molecule should retain or have the ability to bind to the 3′ UTR of PDE 3B and/or to reduce expression of PDE 3B. Functional truncations or fragments of these sequences (or these substantially homologous or variant sequences) could also be used providing one or more of the various appropriate functional properties as outlined above is present or retained. Again, ultimately and preferably the final single stranded miRNA product or molecule should retain or have the ability to bind to the 3′ UTR of PDE 3B and/or to reduce expression of PDE 3B. Other preferred examples of variant or substantially homologous sequences are sequences containing up to 8, e.g. up to 7, 6, 5, 4, 3, 2, or 1 altered nucleotides, or 1, 2, 3, 4, 5, 6, 7 or 8 altered nucleotides (additions, substitutions, insertions or deletions, preferably substitutions) in the above sequences.

Preferred variant or substantially homologous sequences based on the native miR-142-5p sequence (e.g. based on SEQ ID NO:1) would retain one or more, and preferably all, of the nucleotides corresponding to nucleotides 2, 3, 4, 5, 6, 7, 9, 10, 13, 15, 16, 18 or 19 of the native miR-142-5p sequence disclosed herein (SEQ ID NO:1). These residues have complementarity with the PDE3B 3′UTR and thus are believed to be important in the native miR-142-5p sequence for binding to the 3′UTR, see for example Table 2. In particular, one or more, and preferably all, of the nucleotides corresponding to the seed sequence of native miR-142-5p (nucleotides 2 to 7 of SEQ ID NO:1) are preferably retained. In other words one or more, or preferably all, of the nucleotides corresponding to nucleotides 2, 3, 4, 5, 6 and 7 of the native miR-142-5p sequence (see SEQ ID NO:1) are preferably retained.

The variant or substantially homologous sequences also includes modifications or chemical equivalents of the nucleotide sequences used in the present invention that perform substantially the same function as the nucleic acid molecules used in the present invention in substantially the same way. For example, any substantially homologous sequence should retain one or more of the functional properties as described above. Preferably, any substantially homologous sequence should retain one or more (or all) of the functional properties of the starting nucleotide sequence.

A person skilled in the art will appreciate that appropriate assays, e.g. binding assays, can be used to test or assess whether variant or substantially homologous molecules have the chosen functional properties. For example 3′UTR binding assays, or in other words assays to determine the ability of a nucleotide sequence to bind to a 3′ UTR sequence, in this case the 3′ UTR of PDE3B, in particular the sequence of PDE 3B, preferably human PDE 3B, as shown in Table 2, would be well known and standard to a person skilled in the art, as would assays to determine the effect on expression levels of PDE3B.

miRNAs are a good length for therapeutic applications as dsRNAs longer than 30 nucleotides can activate the IFN pathway. Thus, where modified or synthetic or non-native miRNA molecules or sequences are used, the final single-stranded miRNA molecules which will be used to inhibit gene expression, are preferably up to 30 nucleotides in length, e.g up to 29, 28, 27, 26, 25, 24, 23, 22, 21, 20, 19 or 18 nucleotides in length. Other preferred lengths are molecules which are at least 15, 16, 17, 18, 19, 20, 21, 22, 23 or 24 nucleotides in length, or molecules which are up to 25, 26, 27, 28, 29 or 30 nucleotides in length. Preferred lengths might be 17, 18, 19, 20 or 21 to 25 or 26 nucleotides.

The design of therapeutic miRNA molecules for use in the present invention should preferably be such that the nucleotide sequence should be almost, if not entirely, identical to the endogenous miRNA of interest. However, in some embodiments, e.g. embodiments where miR-142-5p is not administered within Tregs, e.g. is itself administered as an active agent, e.g. administered directly as the active agent, as discussed elsewhere herein, non-sequence-based modifications, for example chemical modification, may be desirable to increase stability or to render the miRNAs unrecognisable by the host immune system so that they do not trigger an immune response. The use of the same sequence as the endogenous miRNA is preferred to help avoid this problem. Thus, preferred miR-142-5p for use in the invention comprise or consist of the native miR-142-5p sequence, i.e. CAUAAAGUAGAAAGCACUACU (SEQ ID NO:1), or a near native or essentially native sequence, although other sequence variants may also be used, for example as discussed elsewhere herein.

In the Tregs of this aspect of the present invention, the level of miR-142-5p (or a variant thereof) is increased. Thus, where level of miR-142-5p is discussed herein, then such a discussion can equally refer to variant molecules. Such increases can be carried out in any appropriate way which would be well known to a person skilled in the art. Preferred methods might include gene (or polynucleotide or DNA) insertion, e.g. via lentivirus or other viral vectors, or via well described techniques of gene editing such as CRISPR and other techniques such as base editing, zinc finger nucleases or Transcription activator-like effector nucleases (TALENs), to for example introduce additional copies of the miR-142-5p gene (or additional copies of a gene encoding miR-142-5p).

Thus, such additional (or further) copies of a gene (or polynucleotide or DNA) encoding miR-142-5p could comprise the native miR-142-5p sequence or a variant thereof as described elsewhere herein. Such additional copies could comprise a full length gene or sequence encoding miR-142-5p, or comprise a gene or sequence encoding a precursor of miR-142-5p as described elsewhere herein, or comprise a gene or sequence encoding the native miR-142-5p sequence (SEQ ID NO:1) or a variant thereof, for example encoding only the native miR-142-5p sequence (SEQ ID NO:1) or a variant thereof. In other words, in some embodiments the native miR-142-5p sequence or a variant thereof, or a sequence comprising said sequence, might be the only part of the gene or sequence encoding miR-142-5p that is increased, for example a sequence encoding miR-142-3p may not necessarily be present.

Indeed, in some embodiments of the invention, it may be desired to only upregulate or increase miR-142-5p (or variant) levels, e.g. expression levels. For example, in such embodiments miR-142-3p molecules would not necessarily be present, or not upregulated or increased or overexpressed, e.g. because the miR-142-5p producing molecules are designed so that miR-142-3p molecules are not expressed, or for example levels of such miR-142-3p molecules are inhibited or reduced or decreased or removed, for example using any appropriate method. Standard methodology can be used to carry out such inhibition, reduction, decreasing or removal. For example, appropriate methods will generally be based on antisense approaches or antisense-like approaches in which synthetic single stranded RNAs can act as miRNA antagonists in order to inhibit the action of or degrade the miRNAs, in this case miR-142-3p. These are also known as antagomirs or anti-miRs.

Thus, a yet further aspect of the invention provides Tregs with increased or reduced (decreased) levels (as appropriate), e.g. expression levels, of miR-142-5p, e.g. only miR-142-5p. For example, Tregs have normal, wild-type or endogenous levels of miR-142-3p, or, put another way, miR-142-3p levels are not increased or reduced (decreased) or not significantly increased or reduced (decreased) in the Tregs (as appropriate, depending on the embodiment), e.g. are unchanged, for example compared to an appropriate control such as an unmodified Treg. For example, miR-142-3p levels are not affected (or not significantly affected). In other examples, Tregs have reduced or knockdown levels of miR-142-3p, or miR-142-3p is not present (or not expressed). For example, miR-142-3p can be removed or reduced, e.g. cytoplasmic levels or nuclear levels can be removed or reduced using standard and appropriate methods, e.g. knockout, deletion or knockdown of endogenous miR-142-3p gene or encoding polynucleotide sequences, or using antisense techniques as described elsewhere herein. Alternatively, the polynucleotide sequences used to increase (or reduce) the levels of miR-142-5p (or variants thereof) do not contain miR-142-3p sequences.

Conveniently, the expression level of miR-142-5p is increased or upregulated. Put another way miR-142-5p is overexpressed. Such increase in level or expression level can thus take place by increasing endogenous expression of the miR-142-5p from the genome of the Treg cell, for example by up regulating promoter activity or the activity of other entities or regulatory elements involved in positive regulation of the expression of miR-142-5p, or by reducing repressor activity or the activity of other entities or regulatory elements involved in negative regulation of the expression of miR-142-5p.

The term “endogenous,” when used in reference to a polynucleotide such as miRNA or a gene, refers to a native polynucleotide or gene in its natural location in the genome of an organism. Thus, in this case, endogenous expression of miR-142-5p refers to the expression of miR-142-5p from the native genomic sequence which encodes it.

Alternatively, such an increase in level or expression level of miR-142-5p might be achieved by recombination techniques, e.g. homologous recombination techniques, for example using CRISPR or AAV mediated homologous recombination, to insert a copy of a gene (or polynucleotide) encoding miR-142-5p into the native locus of the miR-142-5p gene. Thus, the inserted gene (or polynucleotide) would replace the endogenous gene and prevent or significantly prevent endogenous expression. Increased level or expression of miR-142-5p from the integrated sequence could however be achieved using an appropriate promoter sequence or other regulatory sequence, e.g. a heterologous promoter or regulatory sequence, to promote expression of miR-142-5p. As described elsewhere herein, inducible or constitutive promoters could be used, which are well known in the art. Constitutive promoters would generally be preferred.

Alternatively, such an increase in level or expression level of miR-142-5p might be achieved by inserting one or more further copies of the nucleotide sequence encoding miR-142-5p into the Treg. Such insertion can be carried out by methods known in the art, for example the additional sequences may be supplied on expression vectors or expression constructs, e.g. by transfection. Such additional copies are thus heterologous copies or exogenous copies. The additional or further copies of the sequences encoding miR-142-5p are conveniently supplied using appropriate plasmids, vectors or expression cassettes which can become integrated into the genome/chromosome of the host cell or can remain non-integrated/extra-chromosomal. However, preferred methods involve the insertion or integration of the sequence encoding miR-142-5p into the genome (also referred to as transformation) which generally involves the use of appropriate viral vectors which can integrate into the genome.

Thus, the present invention also provides a Treg cell, preferably a recombinant Treg cell, comprising a polynucleotide encoding miR-142-5p, preferably a heterologous polynucleotide encoding miR-142-5p. Preferably said polynucleotide or heterologous polynucleotide is integrated into the genome or chromosome of the Treg cell. Depending on the vector used, the integration can be random (e.g. when vectors such as lentivirus vectors are used) or targeted to particular sites in the genome or chromosome (e.g. when homologous recombination vectors are used). As described above, a preferred site of integration would be the site of the endogenous polynucleotide (or gene) encoding miR-142-5p.

More specifically, the invention provides recombinant Tregs having one or more integrated polynucleotides encoding a miR-142-5p miRNA.

A recombinant Treg as used herein refers to a Treg which is produced by recombinant methods, e.g. recombinant DNA techniques or molecular cloning, and is a Treg which is not native, or does not correspond to native or naturally occurring Tregs or Tregs found in nature. For example, the Treg is modified as compared to its native state, for example is genetically modified or genetically engineered. Such recombinant Tregs thus differ in some way from Tregs found in nature or native Tregs.

Thus, modified Tregs (or non-native or non-natural Tregs), for example recombinant Tregs or genetically modified or genetically engineered Tregs, form preferred aspects of the invention. Such modified Tregs can have increased or decreased (as appropriate) levels of miR-142-5p or a variant thereof. In other words, the Tregs are modified (or have been modified) such that the levels of miR-142-5p or a variant thereof are increased or decreased (as appropriate). Such modified Tregs can conveniently be isolated Tregs, or in vitro/ex vivo Treg preparations.

Any appropriate method of modification, e.g. genetic modification or genetic engineering, which is suited to increasing the level of miR-142-5p in a Treg cell can be used, examples of which are as described elsewhere herein. Preferably however, lentiviral vectors or well described techniques of gene editing can be used such as CRISPR and other techniques such as base editing, zinc finger nucleases or Transcription activator-like effector nucleases (TALENs).

Such steps of modification, e.g. genetic modification (or genetic engineering) are thus carried out in preferred embodiments of the invention, for example to generate the Tregs of the invention, e.g. the recombinant Tregs of the invention, for example for use in the therapeutic methods of the invention.

Viral vectors encoding miR-142-5p can be used to introduce, administer or transfect miR-142-5p into Tregs and to induce increased levels or expression levels of miR-142-5p and in turn gene silencing or reduced expression or reduced levels, in this case of PDE3B. Viral vectors have the advantage of extremely high transfection efficiency. In addition, the use of viral vectors is particularly preferred as this will allow integration of the DNA encoding the miR-142-5p into the genome of the Treg. Such genomic or chromosomal integration has the advantage of being stable and also inheritable. Thus, allowing long lasting, stable and long-term expression of the miR-142-5p and reduced expression of PDE3B in the Tregs.

This is particularly helpful for cell therapy, e.g. adoptive cell therapy, or autologous cell therapy, or ex vivo methods such as those contemplated by the present invention, where T cells are conveniently taken from subjects, after which Tregs are isolated, modified to express the miR-142-5p or otherwise increase the level of miR-142-5p, and then expanded in vitro, before returning to a subject/patient.

Suitable viral vectors for this purpose would be well-known to a person skilled in the art. For example, viruses that are commonly employed for this purpose include lentiviruses, adenoviruses, and adeno-associated viruses (AAV's). Lentiviruses are particularly preferred as they have been approved for use in vivo. Such viral vectors are chosen as they are extremely efficient in transferring RNA encoding sequences or vectors, in this case miR-142-5p encoding sequences or vectors into the nucleus and preferably into the genome of mammalian Treg cells, preferably human Treg cells, to ensure high levels of expression of RNA, here miR-142-5p.

Vectors are transfected into the cells and the DNA may be integrated into the genome, e.g. by homologous recombination or other methods in the case of stable transfection, or the cells may be transiently transfected. Other examples of mammalian expression vectors include the pSV and the pCMV series of plasmid vectors, vaccinia and retroviral vectors, as well as baculovirus. Any convenient method or vector can be used.

The plasmid, vector or cassette is generally engineered to contain regulatory sequences appropriate for the selected host cell, e.g. Treg, that act as enhancer and/or promoter regions and lead to efficient transcription of the gene, e.g. the gene or sequence encoding miR-142-5p, carried on the expression vector. The goal of a well-designed expression vector for the present invention is the efficient transcription and production of miR-142-5p miRNA.

The promoter initiates the transcription and is therefore the point of control for the expression of the cloned gene or sequence, e.g. miR-142-5p. The promoters used in vectors, e.g. expression vectors, can be inducible, meaning that miR-142-5p synthesis is only initiated when desired by the introduction of an appropriate inducer such as IPTG. miR-142-5p expression however may alternatively be constitutive (i.e. miR-142-5p is constantly expressed, or expressed most of the time) by use of appropriate constitutive promoters in the vectors, e.g. expression vectors, or in the inserted sequences. In some embodiments, the use of a constitutive promoter is preferred.

The term “promoter” thus refers to a nucleic acid sequence capable of controlling the expression of a coding sequence or functional RNA, in this case a functional RNA in the form of miRNA, in particular miR-142-5p. In general, the miRNA coding sequence is located 3′ to a promoter sequence. Exemplary promoters are well known and described in the art. For example, promoters may be derived in their entirety from a native gene, or be composed of different elements derived from different promoters found in nature, or even comprise synthetic nucleic acid segments. It is understood by those skilled in the art that different promoters may direct the expression of a gene under different conditions, for example in response to different environmental or physiological conditions. Promoters which cause a gene to be expressed in most cell types at most times are commonly referred to as “constitutive promoters”. “Inducible promoters,” on the other hand, cause a gene to be expressed when the promoter is induced or turned on by a promoter-specific signal or molecule.

All the above methods for use in the present invention can result in increased levels or increased expression levels of miR-142-5p being produced in the nucleus of the Treg, which is then exported or transported to the cytoplasm for further processing, typically using normal cell machinery. Thus, increased levels of a molecule encoding miR-142-5p (e.g. a precursor of miR-142-5p) in the cytoplasm are observed.

The increased levels or increased expression levels of miR-142-5p eventually results in translational repression or mRNA degradation/inhibition and produces a gene silencing effect as described elsewhere herein. The present inventors have found that miR-142-5p leads to reduced expression or gene silencing of PDE3B. Thus, when increased levels or increased expression levels of miR-142-5p (or variants thereof) are provided in accordance with the present invention, for example in the cytoplasm of a Treg cell, levels of PDE3B (levels of PDE3B expression) are reduced, for example levels of PDE3B mRNA are reduced or PDE3B protein expression is reduced or silenced.

Conversely, in embodiments of the invention when decreased levels or decreased expression levels of miR-142-5p (or variants thereof) are provided, for example in the cytoplasm of a Treg cell, levels of PDE3B (levels of PDE3B expression) are increased, for example levels of PDE3B mRNA are increased or PDE3B protein expression is increased.

The term “heterologous” when used in reference to a polynucleotide or a gene, etc., refers to a polynucleotide, gene, etc., not normally found in the host cell (e.g. Treg cell). “Heterologous” also includes a native coding region, or portion thereof, that is reintroduced into the host cell in a form that is different from the corresponding native gene, e.g., not in its natural location in the host cell's genome. The heterologous polynucleotide or gene may be introduced into the host cell by, e.g., gene transfer. A heterologous gene may include a native coding region with non-native regulatory regions that is reintroduced into the native host cell. For example, a heterologous gene can include a native coding region that is a portion of a chimeric gene including non-native regulatory regions that is reintroduced into the native host cell. Such heterologous sequences including a native coding region with non-native regulatory regions, can also be inserted into the native or natural location in the host cell genome, for example where techniques of homologous recombination are used as described elsewhere herein. A polynucleotide (whether a native or non-native polynucleotide) integrated into the genome or a chromosome as described herein is considered a heterologous polynucleotide.

The term “expression”, as used herein, can refer to the transcription and preferably stable accumulation of miR-142-5p. The term “overexpressed”, “overexpression”, “increased expression”, “increased level” (or equivalent terms) as used herein, can refer to expression that is higher than or increased, e.g. measurably or significantly increased, as compared to endogenous expression of the same entity, e.g. miR-142-5p. A heterologous gene or polynucleotide, e.g. encoding miR-142-5p, is thus overexpressed if its expression is higher than or increased, e.g. measurably or significantly increased, as compared to that of a comparable endogenous gene. The term overexpression, etc., can refer to an increase in the level of nucleic acid in a host cell, e.g. a Treg cell, as compared to a control host cell, e.g an appropriate control host cell such as an unmodified (or wild-type or parent) host cell, e.g a Treg cell. Thus, overexpression can result from increasing the level of transcription of the endogenous sequence, e.g. miR-142-5p, in a host cell, e.g. Treg cell, or can result from the introduction of a heterologous sequence, e.g. miR-142-5p, into a host cell, e.g. a Treg cell. Overexpression can also result from increasing the stability of a nucleic acid sequence, e.g miR-142-5p.

As used herein the term “transformation” refers to the transfer of a nucleic acid fragment, e.g. a polynucleotide encoding miR-142-5p, into the genome of a host cell, e.g. Treg, resulting in genetically stable inheritance. Host cells containing the transformed nucleic acid fragments can be referred to as “transformed” cells.

The terms “plasmid”, “vector” and “cassette” can refer to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product, in this case a DNA sequence encoding miR-142-5p, optionally along with appropriate 3′ untranslated sequences into a host cell, e.g. a Treg. “Transformation vectors” refers to a specific vector containing a heterologous polynucleotide, e.g. miR-142-5p, and having elements in addition to the polynucleotide, e.g. miR-142-5p, that facilitates transformation (integration into the genome) of a particular host cell, e.g. Treg. “Expression vectors” refers to a specific vector containing a heterologous polynucleotide, e.g. miR-142-5p, and having elements in addition to the heterologous polynucleotide, e.g. miR-142-5p, that allow for enhanced expression of that gene in a host cell, e.g. Treg.

Alternatively, the level of miR-142-5p can be increased in the cytoplasm of the Tregs by for example transfecting the cells directly with miR-142-5p molecules. Such methods (non-recombinant methods) for transfecting RNA molecules, including miRNA molecules are well known in the art.

For such direct transfection methods, synthetic miRNAs (also known as miRNA mimics) can conveniently be used to mimic the function of endogenous miRNAs. This approach also leads to translational repression and/or mRNA degradation/inhibition and produces a gene silencing effect.

Synthetic miRNA are aimed to achieve the same biological functions as the endogenous or native miRNA. Thus, ideally they should possess the ability to be processed by the intracellular processing machinery for endogenous or native miRNAs, such as the Dicer protein, and/or to be loaded into or interact with the RISC and therefore silence the target mRNAs through the natural miRNA pathway, in this case by ultimately being able to bind to the 3′ UTR of PDE3B and/or to reduce levels or expression of PDE3B.

Thus, for miR-142-5p, a single stranded RNA molecule containing (e.g. comprising or consisting of) the miR-142-5p sequence (CAUAAAGUAGAAAGCACUACU, SEQ ID NO:1) that is identical to the guide strand of the mature duplex miRNA can be used as such a molecule should be able to bind to the target mRNA, in this case the 3′UTR of the PDE3B target mRNA and reduce or silence gene expression. Fragments or variants of the sequence might equally be used as described elsewhere herein, providing that the ability to bind to the target mRNA, in this case the 3′UTR of PDE3B, is present or retained, and/or the ability to reduce levels or expression of PDE3B is present or retained.

However, double stranded forms of miRNA containing both guide and passenger strands, including miRNA duplexes and hairpin structures such as pre-miRNA molecules which contain sequences encoding both guide and passenger strands, are thought to be more potent and preferred molecules to use. The double-stranded structure can facilitate the proper processing of the RNA molecules by the intracellular processing machinery for endogenous or native miRNAs, such as the Dicer protein, and/or to be loaded into or interact with the RISC, thereby potentially enhancing the gene silencing effect. Thus, preferred miRNA mimics have a duplex RNA structure or a hairpin structure. Preferably the sequence of the duplex structure or hairpin will correspond to the native sequence of miR-142 as described elsewhere herein and contain both miR-142-5p and miR-142-3p sequences. However, fragments or variants of these sequences might equally be used as described elsewhere herein, providing that e.g. the ability to be processed by the intracellular processing machinery for endogenous or native miRNAs, such as the Dicer protein and/or to be loaded into or interact with the RISC, and ultimately to bind to the target mRNA, in this case the 3′UTR of PDE3B, is present or retained, and/or the ability to reduce levels or expression of PDE3B is present or retained.

Thus, synthetic miRNA molecules with longer sequences than the mature miR-142-5p/miR-142-3p miRNA duplex (from a few extra nucleotides to a pre-miRNA or a full length pri-miRNA) can be used. Since pri-miRNA require processing in the nucleus (unlike pre-miRNA or miRNA), these sequences or molecules require a different mode of delivery such that they can locate to the nucleus. For example, appropriate vectors, preferably viral vectors, as described elsewhere herein can be used to target and express such miRNAs inside the nucleus of cells as opposed to simply providing miRNA directly into the cytoplasm. Again, fragments or variants of these sequences might equally be used as described elsewhere herein, providing that the ability to be processed by the nuclear processing machinery, such as Drosha, the ability to be exported or transported to the cytoplasm, e.g. via Exportin 5, the ability to be processed by the intracellular processing machinery for endogenous or native miRNAs, such as the Dicer protein, and/or the ability to be loaded into or interact with the RISC, and ultimately the ability to bind to the target mRNA, in this case the 3′UTR of PDE3B, is present or retained, and/or the ability to reduce levels or expression of PDE3B is present or retained.

Regulatory T cells (Tregs) as referred to herein, are a subpopulation of T cells, e.g. a subpopulation of CD4 expressing T cells, which modulate the immune system, maintain tolerance to self-antigens, and abrogate autoimmune disease. Tregs thus have a key role in immune suppression and are the master controllers of self-tolerance, tissue inflammation and long-term immune homeostasis. They can also suppress or down regulate induction and proliferation of effector T cells (Teffs). Regulatory T cells come in many forms with the most well-understood being those that express CD4, CD25, and Foxp3.

Any type of Treg cell may be used in the invention, for example thymic tTregs or nTregs (which can for example be obtained from any appropriate source, including peripheral blood), or inducible Tregs (iTregs), which can be produced or induced in vitro from CD4+ T cells (naïve CD4+ T cells) using standard methods, e.g. using molecules such as IL-2 and TGF-beta,

Thus, one appropriate and preferred marker panel for Treg cells is CD4+CD25+ (or CD25 high) FOXP3+. CD25 is a gene that is expressed largely by lymphocytes and to a particularly strong extent by Tregs. Thus, Tregs (Treg cells) for use in the present invention may be characterized by the expression of CD4, CD25 and FoxP3, and preferably CD127− (or CD127 low). Another marker panel for Treg cells is CD3+, CD4+, CD25+, FOXP3+ and CD45+. Thus, Tregs (Treg cells) may be characterized by the expression of CD3, CD4, CD25, FoxP3 and CD45, preferably CD45RA. Thus, CD45RA is a further marker which can be expressed by the Tregs for use in the invention. Treg cells are also generally CD127− (or CD127 low).

Although other markers may be expressed, Tregs for use in the present invention should retain FOXP3 expression and a functional ability to suppress activation of Teffs. In addition, the Tregs of the invention and for use in the present invention are Tregs (modified Tregs) which, in one embodiment, have increased levels of miR-142-5p as described elsewhere herein and which preferably results in an improved or increased functional ability or activity, in particular to suppress or reduce activation or function of Teffs. In other embodiments, the Tregs of the invention and for use in the present invention are Tregs (modified Tregs) which have decreased levels of miR-142-5p as described elsewhere herein, and which preferably results in a reduced functional ability or activity, in particular a reduced ability to suppress or reduce activation or function of Teffs. Thus, Tregs of or for use in the present invention are preferably modified, e.g. genetically modified or genetically engineered, for example to have increased (or decreased, as appropriate) levels of miR-142-5p. In addition, Tregs of or for use in the present invention are preferably taken from or obtained from subjects with autoimmune (AI) disease, or cancer, as appropriate.

Tregs for use in the invention can be obtained from any appropriate source, although the use of blood samples, e.g peripheral blood (in particular PBMCs) is particularly convenient. As preferred embodiments of the therapeutic methods of the invention are methods of cell therapy (T cell therapy), for example methods of adoptive cell therapy (ACT), conveniently said methods use autologous T cells which can then be manipulated or treated ex vivo/in vitro and used for autologous therapy. In other words, the Tregs are taken from the patient/subject that is to be treated (e.g. subjects with AI disease or cancer, as appropriate), modified (e.g. by gene editing or genetic engineering), expanded, and then returned to the same patient for therapy. However, non-autologous adoptive cell therapies are also contemplated, for example where steps can be taken to minimise or remove potential issues with cell rejection. Isolated Tregs, or in vitro/ex vivo preparations of Tregs, thus form preferred embodiments of the invention.

Populations, e.g. populations of T cells (e.g. populations of Tregs), which have been selected or isolated from a subject, i.e. are no longer present in situ within the subject, are conveniently used to produce the modified Tregs of the invention and in the therapeutic uses of the invention. Preferably such T cell populations (Tregs) are pure (or relatively pure), e.g. within the bounds of experimental practice and protocols, and contain minimal numbers of other cell types. For example, the number of other cell types is at a low enough level so that the population as a whole functions as a pure (or relatively pure) population of Tregs, and the presence of the other cell types does not affect or significantly affect the function of Tregs. Put another way, the predominant cell type in the population is Tregs, for example at least 70%, 80%, 90%, 95%, or 98% of cells are Tregs. Thus, a yet further aspect of the invention provides populations of Treg cells of the invention (modified Tregs) as described elsewhere herein.

Appropriate methods for obtaining Tregs from a patient or subject would be well known and standard to a person skilled in the art. For example, a blood sample can be taken from the patient and the Treg subpopulation can be isolated by standard methods, for example by taking PBMCs and using marker panels as described elsewhere herein in methods such as FACS cell sorting. Methods for preparing populations of inducible Tregs, tTregs or nTregs are also well described in the art in embodiments where these are to be used. Such isolated or selected T cells or T cell populations (isolated or selected Treg cells or Treg populations) are preferred for the therapeutic uses of the invention and for administration to subjects to be treated.

In preferred therapeutic methods of the invention, the population of T cells is expanded before being administered to the subject. Preferred methods thus comprise such an expansion step. Appropriate methods, e.g. in vitro or ex vivo methods, for the expansion of Tregs are also well known in the art and any of these can be used. For example, Tregs can be purified or isolated from the peripheral blood (or other appropriate T cell containing sample, e.g. umbilical cord blood) of a subject, for example using marker panels as described elsewhere herein, grown ex vivo/in vitro, for example in the presence of antibodies to CD3 and/or CD28 accompanied by IL-2, e.g. high-dose IL-2, to expand the highly enriched Treg population, and, after adequate characterisation, transferred, e.g. adoptively transferred, into subjects. Preferred methods of in vitro/ex vivo expansion might additionally involve expansion in the presence of rapamycin, which can help to prevent growth of contaminating Teffs in Treg cultures and can enhance phenotypic stability. Retinoic acid may also be present (or other appropriate methods used) to increase alpha 4 beta 7 integrin expression levels, which can improve gut homing of the T cells.

Such polyclonal Treg adoptive cell therapy has been shown to be safe in phase I studies Equally, the invention contemplates the use of non-polyclonal Treg populations, e.g. monoclonal Tregs or antigen-specific Tregs.

Some embodiments of the invention involve Treg enrichment on the basis of CD45RA+, in particular CD4+, CD25+/high, CD127−/low, FOXP3+ and CD45RA+. A particularly convenient method of expansion is described in Canavan et al. (Gut 65:584-594, 2016). For example, an initial enrichment on the basis of CD45RA+ is shown to generate a homogenous and epigenetically stable Treg population following expansion, in the presence of rapamycin, from the peripheral blood of subjects. This Treg population has been shown to be resistant to TH 17 plasticity, to express lymphoid and gut homing markers, and home to human gut.

In vitro expansion also enhances the suppressive ability of these cells for Teffs, making the CD4+, CD25+/high, CD127−/low, FOXP3+ and CD45RA+ Tregs an appropriate population from which to expand Tregs in vitro for use in cell therapy. Preferably said in vitro expansion step will involve the presence of IL-2, e.g. high-dose IL-2, rapamycin and anti-CD3/anti-CD28 (for example using anti-CD3/anti-CD28 beads).

In some embodiments of the invention, gut homing or other tissue or organ targeting techniques can be used in order to direct or target modified Tregs of the invention, e.g. miR-142-5p containing Tregs of the invention, to the correct and desired body location to be effective. For example, if a target antigen is present in the target tissue or organ, e.g. there is a tumor or gut or skin or liver specific antigen, then the Tregs can be targeted to these areas using techniques which can target such antigens, such as using CARs. For example, the Tregs could be engineered to express such CARs to target these areas.

Other methods for tissue or organ homing, e.g. to the tissues or organs outlined above, are known and may be used. For example, naturally occurring markers which can target cells to particular organs or tissues are known. For example, there are known T cell markers, e.g. as described above, which can target T cells to the gut. Thus, if T cells, in this case Tregs, can be selected which express such markers then gut homing can be improved which should also then aid efficacy when the disease to be treated is for example a gut associated autoimmune disease. This increased ability to home to the gut will be advantageous for the treatment of autoimmune diseases of the gut, in particular IBD, including UC and CD.

Once an appropriate Treg or population of Tregs has been obtained, the cells are then modified as described elsewhere herein e.g. by genetic modification or genetic engineering, in order that the level of miR-142-5p (CAUAAAGUAGAAAGCACUACU, SEQ ID NO:1) or a variant thereof is increased.

The term “increase” or “enhance” or “overexpression” (or equivalent terms) as described herein in relation to the level or level of expression of miR-142-5p in a Treg cell (or population of Treg cells) includes any measurable increase or elevation when compared with an appropriate control. Appropriate controls would readily be identified by a person skilled in the art and would include the level of miR-142-5p in an unmodified Treg cell (or population of unmodified Treg cells), e.g. the level of endogenous expression in a Treg cell, e.g. a Treg which had not been subject to modification, e.g. genetic engineering or modification, to increase the level of miR-142-5p. As described elsewhere herein, such unmodified Tregs could be those from a subject with AI disease, e.g. a subject to be treated in accordance with the present invention (e.g. a subject with impaired or abnormal Treg suppressor function and possibly reduced levels of miR-142-5p compared to healthy subjects), or a comparison could be made with levels of miR-142-5p in Tregs from a population of such subjects, e.g. a comparison could be made with predetermined levels of miR-142-5p. Preferably any such increase in levels of miR-142-5p will also result in a reduction (or decrease), e.g. a measurable or significant reduction (or decrease) in the levels or expression of PDE3B, and/or an increase, e.g. a measurable or significant increase in intracellular cAMP levels, e.g. when compared to the appropriate control. Preferably the increase (or equivalent), or decrease (as appropriate) will be significant, for example clinically or statistically significant.

Viewed another way, preferably the increase (or equivalent) in level, e.g. expression level, of miR-142-5p (or intracellular cAMP), or decrease (as appropriate) of PDE3B, is functionally significant, for example is an increase (or decrease) to a level such that the ability of the Tregs to suppress Teffs is increased, preferably significantly increased, preferably a statistically or clinically significant increase. The ability of Tregs to suppress Teffs can be measured using any appropriate assay. A convenient assay to measure this ability is an in vitro co-culture suppression assay, e.g. as described in the experimental Examples, in which appropriate populations of Tregs and Teffs are co-cultured at different ratios, and the suppression (e.g. percentage suppression) of proliferation is calculated using an appropriate technique (e.g. using the formula as disclosed in the experimental Examples). For example, when a 1:1 ratio is used, wild-type Tregs have a percentage suppression of proliferation of Teffs of approximately 50 to 60%. Thus, preferably the increase in level of miR-142-5p (or intracellular cAMP), or decrease (as appropriate) of PDE3B, is such that the modified Tregs have a percentage suppression of proliferation of Teffs that is increased, preferably significantly or statistically significantly increased, for example compared to wild-type or unmodified T cells, e.g. when a 1:1 ratio is used. Alternatively, for example, the increase in level of miR-142-5p, etc., is such that the modified Tregs have a percentage suppression of proliferation of Teffs that is at least 55%, 60%, 65%, 70%, or 80%, for example when a 1:1 ratio is used.

Thus, a yet further aspect of the invention provides a method for preparing (or producing) Tregs suitable for use or for use in the treatment of AI disease, said method comprising the following steps:
i) Isolating Tregs from a sample taken from a subject, preferably a blood sample;
ii) Modifying the Tregs, e.g. by genetic modification or genetic engineering, so that the level of miR-142-5p (CAUAAAGUAGAAAGCACUACU, SEQ ID NO:1) or a variant thereof is increased; and optionally
iii) in vitro expansion of the Treg cells.

Appropriate methodology for carrying out the above steps is described herein or would be well known to a person skilled in the art. The steps may be carried out in any appropriate order, although preferably the steps are carried out in the order shown. For example, conveniently the expansion step can be carried out after the modification step, in particular where said modification is a stable or inheritable modification. In some embodiments however, for example where said modification is not a stable or inheritable modification, the expansion step could be carried out before the modification step. Preferably all of the steps will be carried out, although in some embodiments, the Tregs for modification may be obtained from a different source or may have already have been isolated in which case an appropriate step would for example involve modifying isolated Tregs or simply modifying Tregs before an optional expansion step.

A yet further aspect of the invention provides a population of Tregs (modified Tregs) produced, obtained or obtainable by the above methods.

The present invention is based on the finding by the inventors that miR-142-5p can directly target and reduce the expression of phosphodiesterase-3B (PDE3B), e.g effect or induce gene silencing of PDE3B. In this regard, the inventors have shown that miR-142-5p can bind to or target the 3′ UTR of PDE3B.

PDE3B is a cAMP hydrolysing enzyme and the present inventors have shown that reduced expression of PDE3B in Tregs in turn results in increased expression of intracellular cyclic AMP (cAMP) and increased or enhanced activity of Tregs. One of the manifestations of this increased or enhanced activity of Tregs is the increased or enhanced ability to suppress T effector cells (Teffs), e.g by reducing or inhibiting or blocking the proliferation of Teffs. This increased activity of Tregs and enhanced suppression (or reduced activity) of Teffs induced by increasing the level of miR-142-5p and the reduced expression of PDE3B, means that such Tregs can be used in the treatment of autoimmune (AI) diseases. AI diseases generally involve hyperactive or overactive or dominant (e.g. dominant numbers of) Teffs and/or underactive or insufficient numbers of Tregs.

Thus, the present invention further provides therapeutic methods for AI diseases.

Thus, at its broadest the present invention provides an agent which inhibits or reduces PDE3B levels (e.g. protein or mRNA levels), or activity, for use in the treatment of AI diseases. Preferably the agent is one that inhibits or reduces PDE3B levels (e.g. protein or mRNA levels), for example a preferred such agent is miR-142-5p. Preferably the agent is selective for inhibition of PDE3B (e.g. inhibition or reduction of PDE3B levels), for example said agent is preferably selective for inhibition of PDE3B (e.g. inhibition or reduction of PDE3B levels) over the inhibition of PDE3A, for example the agent preferably or preferentially inhibits or reduces PDE3B as compared to PDE3A (for example, the inhibition or reduction of PDE3B is measurably, and preferably significantly, higher than the inhibition or reduction of PDE3A), even more preferably PDE3A is not inhibited or reduced (e.g. is not measurably inhibited or reduced) or is not significantly inhibited or reduced by the agent. Again a preferred such agent is miR-142-5p where it can be seen that the mode of action of miRNA, i.e. by sequence-based interaction with the 3′UTR of a target gene, in this case PDE3B, lends itself to such selective action. Preferably the inhibition or reduction in PDE3B levels or activity is in Tregs. Preferably such treatment allows mechanisms of tolerance in subjects suffering from AI to be re-established.

Thus, in a further aspect the present invention provides miR-142-5p, for example miR-142-5p (CAUAAAGUAGAAAGCACUACU, SEQ ID NO:1) or a variant thereof, for use in the treatment of AI diseases.

Thus, a yet further aspect of the invention provides miR-142-5p in a formulation or format suitable for therapeutic use or administration to a subject/patient. Appropriate formulations are described elsewhere herein and include for example nanoparticles and liposomes comprising miR-142-5p.

Other aspects of the invention provide methods of reducing PDE3B expression or levels in a cell, e.g. a Treg cell, using miR-142-5p or a variant thereof (for example by expressing or overexpressing miR-142-5p) as defined herein.

The present invention further provides methods to selectively target PDE3B, for example to target PDE3B over PDE3A, using miR-142-5p or a variant thereof as defined herein.

As is clear from the above, the present invention relates to the types of T cell known as effector T cells (Teffs) and regulatory T cells (Tregs), which are very important mediators of the immune responses seen in autoimmune diseases. In humans, Tregs are associated with dominant peripheral tolerance, and genetic defects affecting Treg function result in multi-system inflammation, including the intestine. Tregs suppress immune responses in the intestine and over-active Teffs are believed to be the cause of IBD. Similarly, an imbalance, in the relevant tissue or organ, of Tregs and Teffs is believed to be involved in other types of AI disease. Therefore, a critical rate-determining step for whether a patient develops an AI disease is the balance between Teffs and Treg function in the relevant tissue or organ (e.g. the intestine for IBD).

The ratio of Tregs to Teffs (the Treg:Teff or Tr:Te ratio) is relevant for this balance. Healthy patients have a high ratio. Many autoimmune diseases are associated with a low ratio, which generally results in a pro-inflammatory phenotype and causes disease. Thus, in the treatment of autoimmune diseases it is generally desired to increase this ratio and/or increase the dominance or numbers of Tregs or reduce the dominance or numbers of Teffs. In cancer patients on the other hand, it is generally desired to decrease this ratio and/or increase the dominance or numbers of Teffs.

The present invention enables modulation of the Treg:Teff T-cell balance by increasing the activity or function of Tregs, e.g. the suppressive activity of Tregs on Teffs, by modulating (here increasing) the level, e.g. the level of expression, of miR-142-5p in Tregs. The use of such Tregs with increased activity can then be used to push or modify the Treg:Teff ratio in favour of Tregs and thereby increase the Treg:Teff ratio and treat AI diseases, or any other diseases involving an unbalanced Treg:Teff ratio in favour of Teffs over Tregs.

In the methods of the invention, after modification, e.g. genetic modification, to increase levels of miR-142-5p as described herein, these highly active Tregs are conveniently expanded in vitro (or ex vivo), and then administered to a subject in need of treatment. The presence of such highly active Tregs in the subject means that these Tregs show an enhanced ability to suppress activity of Teffs in a subject, e.g. increased ability to reduce proliferation of Teffs in a subject, which can then for example act to decrease numbers of Teffs in the subject (e.g. compared to an untreated subject), rebalance (or increase) the Treg:Teff ratio (e.g. compared to an untreated subject), reduce inflammation (e.g. compared to an untreated subject), and treat the autoimmune disease.

Because the present invention can allow reduced numbers of Teffs to be produced and/or increased numbers of Tregs to be produced and, for example, allow the rebalancing or increase of the Treg:Teff cell ratio and/or a reduction in inflammation, the Tregs and therapeutic methods of the present invention can be used to treat any autoimmune (AI) disease (e.g. any disease associated with abnormal and elevated inflammation and/or abnormal or imbalanced Treg:Teff cell ratio (usually in favour of Teffs over Tregs), or any AI disease characterised by or associated with an excess in Teff number, function or activity, or a deficiency in Treg cell number or function or activity). Exemplary AI diseases that can be treated in accordance with the present invention are IBD, for example Crohn's disease or ulcerative colitis, treatment of other AI diseases of (or affecting) the gut, e.g. autoimmune gastritis, coeliac disease, and colitis/autoimmunity associated with treatment of cancer patients, for example associated with checkpoint inhibitor treatments such as anti-CTLA-4, anti-PD1 or anti-PDL1 treatments.

Other examples of autoimmune diseases which can be treated in accordance with the invention are ankylosing spondylitis, psoriasis, primary sclerosing cholangitis, Type 1 diabetes (T1D), Kawasaki disease, vasculitis (e.g. HCV related vasculitis), SLE, alopecia areata, rheumatic disease, e.g. rheumatoid arthritis (RA), other types of arthritis such as juvenile idiopathic arthritis, hematopoietic stem cell transplantation (HSCT), graft-versus-host disease (GvHD) that can for example occur after bone marrow transplantation, and organ transplant rejection.

At present, the treatment of autoimmune diseases, for example autoimmune diseases of the gut, currently relies on either blanket suppression of the immune system (e.g. corticosteroids and anti-proliferative agents) or selective cytokine blockade of candidate molecules overexpressed in diseased tissue (e.g. anti-TNF antibodies). Thus, new and ideally improved therapeutic options for such diseases as provided by the present invention are clearly desirable.

Even more preferably, the Tregs for use in the treatment of AI diseases express homing markers which are appropriate for the relevant immune niches for the disease in question, e.g. the target (disease affected) tissue or organ, where the Tregs can suppress inflammation. For example, for AI diseases, in particular AI diseases of the gut or mucosa, gut or mucosal homing markers are preferably expressed, such as one or more of alpha 4 beta 7 integrin, CD62L, CCR6, CCR7, and CXCR3. Such T cell populations with homing markers thus preferably have the ability or capability to home to appropriate tissues or organs, e.g. show the ability to home to gut or mucosal tissue. Gut homing markers are especially preferred when the AI disease is an AI disease affecting the gut. Expression of alpha 4 beta 7 integrin and/or CCR6 are particularly useful for gut or intestinal mucosal homing, e.g. homing to the lamina propria (LP), and thus are desired when Tregs are used to treat AI diseases affecting the gut. Expression of CD62L and/or CCR7 are particularly useful for homing to lymphoid tissue, for example intestinal lymphoid tissue such as mesenteric lymph nodes (MLN). Expression of CXCR3 is particularly useful for homing to sites of inflammation, and thus expression of this marker has utility in homing Tregs to the sites of AI disease. Preferred Tregs are also resistant to Th17 conversion, for example do not express Th17 related genes.

Preferred therapeutic agents are modified Tregs of the invention as described herein, e.g. genetically modified or recombinant Tregs, with increased levels of miR-142-5p. Put another way, in preferred embodiments of the present invention, the miR-142-5p molecule is delivered or administered to a subject/patient within a Treg cell, for example by administering a Treg of the invention, e.g. a genetically modified or recombinant Treg in which the level of the miR-142-5p has been increased or overexpressed. The use of Tregs as a delivery system also has the advantage of a much better in vivo stability, for example the use of the Treg cells protects the miRNA, for example allowing the avoidance of issues with nuclease digestion and therefore avoiding or reducing the need for chemical modification. Populations of such Tregs are conveniently provided and used in the present invention.

Tregs of the invention which comprise miR-142-5p molecules can also be used to transfer miR-142-5p molecules to other cells, for example in vivo or in vitro. This transfer, e.g. trans-transfer, can for example take place using the exosome transfer pathway present in the Treg. Such transfer can therefore enhance and add to the intrinsic effects of miR-142-5p molecules in the Tregs by for example transferring miR-142-5p molecules to other cells which may be other Tregs, e.g. endogenous Tregs, or other types of cell, e.g. other types of endogenous cell, in a subject.

However, in other aspects of the invention the level of miR-142-5p can be increased in Tregs in other ways, for example by administering miR-142-5p to subjects using alternative techniques which will allow uptake of miR-142-5p into Tregs. Such methods of administering RNA to subjects, for example for various therapeutic applications, are known in the art and such methods can also be used for administering miRNA.

For example, miRNA molecules can be directly administered and a yet further aspect of the present invention provides miR-142-5p, for example miR-142-5p (CAUAAAGUAGAAAGCACUACU, SEQ ID NO:1) or a variant thereof, for use in the treatment of AI diseases.

RNAs are extremely vulnerable to serum nucleases. Although double-stranded RNA is more resistant to nuclease degradation than single stranded RNA and thus are preferred forms of miRNA for use in these aspects of the invention (e.g. duplex miRNA molecules or pre-miRNA hairpins as described elsewhere herein), naked RNAs in their unmodified forms are degraded rapidly following administration to a subject, e.g. in the bloodstream. Chemical modification of miRNA can be used to address the short half life of miRNA in vivo. In addition, chemical modification of miRNA duplexes can minimise immunogenicity. Any chemical modifications that have previously been shown to work for nucleic acids in general can be applied to miRNA.

Appropriate chemical modification, for example to increase half life in vivo (for example by increasing the resistance to nucleases, e.g. exonucleases) or to minimise immunogenicity would be well-known and routine to a person skilled in the art. However, any chemical modification used should retain the gene silencing ability of the miRNA molecule, for example should retain the ability of the miRNA molecule to bind to mRNA, e.g. the 3′ UTR or other target sequence of the target mRNA, in this case PDE3B, and/or to reduce levels or expression of PDE3B. Preferably the compatibility with other elements of the endogenous miRNA silencing pathway should also be retained, for example the ability to be loaded into the RISC in the correct orientation such that the miR-142-5p is used as the guide strand and the miR-142-3p is discarded as the passenger strand. In general, chemical modifications in the guide strand, and in particular the 5′ end or 5′ proximal region or central region of the guide strand should be avoided as these tend to be the important areas of the RNA duplex for interaction with the RISC and AGO proteins. On the other hand, chemical modifications in the passenger strand are generally better tolerated, as are modifications in the 3′ end or 3′ proximal part of the guide strand. Thus, modifications in these regions are preferred, more preferably in the passenger strand.

Any appropriate modifications may be used, for example modifications known in the art to improve stability of an RNA molecule, in particular an RNA duplex, for example in serum, or to reduce immunogenicity. Exemplary modifications might include ribose 2′-OH group modification (for example by substituting the ribose 2′-OH group with other chemical groups including for example 2′methoxyethyl (2′-O-MOE), 2′-fluoro (2′-F) or 2′-O-methyl (2′-O-Me), phosphorothioate (PS) modification (or other backbone modifications) and/or locked and unlocked nucleic acids. Combinations of these may also be used. 2′-OH group modification, in particular at U residues, with either 2′-fluoro (2′-F) or 2′-O-methyl (2′-O-Me) are particularly preferred for abrogating immune responses without affecting potency. LNA modification is also useful for this. As recognition of an RNA duplex by TLR 7/8 is believed to be a key trigger for the stimulation of an immune response, any modifications which render the RNA duplex unrecognisable by TLR 7/8 or which reduce the recognition of the RNA duplex by TLR 7/8 are preferred. This can be readily tested. Again, modifications of the passenger strand are sometimes preferred.

Any other safe, clinically relevant delivery system that can facilitate uptake of miR-142-5p into target cells, in this case Tregs, and for example offer protection against nuclease degradation can be used.

One appropriate example for miRNA delivery is viral vectors. For therapeutic uses, for example for gene therapy methods such as those described in the present invention, viruses that are used to carry therapeutic miR-142-5p can be genetically engineered to remove their virulence. In addition, and advantageously, it is possible to carry out genetic manipulation of the viral capsid for targeting to specific cell types. This feature is especially helpful in embodiments where the miR-142-5p, for example in the form of viral vectors expressing the miR-142-5p are administered to subjects. For use in the present invention, such viral vectors (or other targeting entities) would preferably be targeted to Tregs, for example using any appropriate methods, for example by exploiting cell surface markers expressed on Tregs and not (or to a lesser extent) on other cells. One such appropriate cell surface molecule might be CD25, which is highly expressed on Tregs but not on Teffs. Thus, for example a complex can be made between an entity which can interact with CD25 and a viral vector (or an miRNA molecule) in order to target the miR-142-5p to Tregs. Again, once the viral vector had been targeted to the cells of interest, in this case Tregs, the use of a viral vector, for example a lentivirus vector, can enable long-term expression by integration into the host genome. Such Treg targeting methods can be used in other embodiments of the invention as appropriate.

Alternatively, non-viral vector systems can be used to deliver miRNA. For example, any appropriate system for nucleic acid, in particular RNA delivery may be used to deliver the miR-142-5p molecules in accordance with the present invention, many examples of which are well known and described in the art.

### Polymer-Based Systems

Cationic polymers can form polyplexes with the negatively charged miRNA. Cationic polyplexes are good for promoting cellular uptake. In addition, the nano-sized polyplexes can facilitate cellular uptake through endocytosis. A preferred example is synthetic polyethylenimine (PEI) which has been used for miRNA delivery in vivo and also shows a high transfection efficiency. Dendrimers, in particular cationic dendrimers such as polypropylenimine or poly(amidoamine) (PAMAM), which are highly branched synthetic polymers are also used for miRNA delivery, as are natural cationic polymers such as chitosan, hyaluronic acid and atelocollagen. Cyclodextrins, cyclic oligomers of glucose, can also be used.

Neutral polymers e.g. poly (lactic-co-glycolic acid), PLGA, is an FDA approved synthetic biodegradable polymer that can be used to deliver miRNA. Such neutral polymers can be made into nanoparticles or microparticles which can be loaded with miRNA as opposed to forming polyplexes. Small amounts of cationic polymers, e.g. PEI, can be incorporated into such particles in order to enhance encapsulation and transfection efficiency. Other nanoparticles such as Silica-based nanoparticles can also be loaded with miRNA and used for in vivo delivery.

### Lipid-Based Systems

Similarly to cationic polymers, cationic lipids and liposomes can form lipoplexes with RNA, e.g. miR-142-5p, through electrostatic interactions. In general, lipids used for nucleic acid delivery are composed of a cationic head group and a hydrophobic chain. Examples of commonly used cationic lipids for nucleic acid delivery are DOTAP and DOTMA, which are often used in combination with neutral lipids such as cholesterol and DOPE to enhance transfection efficiency, and any of these may be used to deliver the miR-142-5p molecules in accordance with the present invention. Equally commercially available lipid-based transfection reagents which are suitable for RNA delivery, such as Lipofectamine, Oligofectamine, etc., may also be used for the delivery of miR-142-5p in accordance with the present invention. Molecules such as PEG may also be incorporated into such lipid particles in order to reduce immunogenicity and increase half life. Lipid-based nanoparticles, e.g. solid lipid nanoparticles, which may also be pegylated, are particularly preferred for miRNA delivery. Exosomal delivery systems may also be used.

Lipolyplexes or lipopolymers, a mixture of both polymers and lipids may also be used. All can incorporate targeting moieties or ligands, such as antibodies and small peptides, to achieve site-specific delivery and to improve the specificity.

All such systems can also be modified to improve serum stability and extend half life. Nanoparticles and liposomes have been used in clinical trials to deliver miRNA and thus are particularly preferred, for example lipid-based nanoparticles or lipid nanoparticles. PEGylated nanoparticles (or other modifications to increase stability or half life) and the incorporation of targeting moieties or ligands are other preferred features.

In addition, as the present inventors have shown that increased levels of miR-142-5p in Tregs has a direct effect on reducing the expression of PDE3B, which in turn increases the expression of intracellular cAMP, and increases the activation status of Tregs, it follows that reduced or decreased levels of miR-142-5p in Tregs will result in increased or upregulated or preserved expression of PDE3B, thereby reducing the expression of intracellular cAMP and reducing or inhibiting the activity of Tregs, in particular reducing the ability to suppress Teffs, for example the Tregs may be less able to inhibit proliferation and/or activation of Teffs. This will lead to increased Teff activation and functionality and to increased numbers and dominance of Teffs. This opens up other therapeutic avenues, namely in cancer therapy, where a pro-inflammatory response is desirable.

Thus, a yet further aspect of the invention provides a T regulatory (Treg) cell in which the level of miR-142-5p (CAUAAAGUAGAAAGCACUACU, SEQ ID NO:1) or a variant thereof is reduced (or decreased).

Such Tregs (modified Tregs) can be used for the treatment of cancer. Thus, the present invention further provides a T regulatory (Treg) cell (modified Treg cell) in which the level of miR-142-5p (CAUAAAGUAGAAAGCACUACU, SEQ ID NO:1) or a variant thereof is reduced (or decreased), for use in therapy, in particular for the treatment of cancer.

In the Tregs of this aspect of the present invention, the level of miR-142-5p (or a variant thereof) is reduced or decreased. Thus, where level (in particular reduced or decreased level) of miR-142-5p is discussed herein, then such a discussion can equally refer to variant molecules. Such reductions or decreases in level, e.g. expression level, can be carried out in any appropriate way which would be well known to a person skilled in the art and will result in modified Treg cells, e.g as described in other aspects of the invention.

Preferred methods for making such modified Tregs might include reducing endogenous (or native) expression of miR-142-5p by any appropriate means, for example by deleting or mutating the endogenous (or native) regulatory elements controlling the expression of miR-142-5p, for example by mutating a transcription factor binding site or enhancer involved in the active expression of miR-142-5p in order to for example reduce or prevent the binding of regulatory proteins such as transcription factors. Examples of transcription factor binding sites are given elsewhere herein, for example it has been shown that the promoter of miR-142 contains separate binding sites for three transcription factors, PU.1, C/EBPb and Runx1 and mutations at one or more of these sites could be used.

Alternative methods would include deleting or mutating the endogenous (or native) genomic sequence encoding miR-142-5p, for example so that transcription of the pri-mRNA is significantly reduced or inhibited, preferably completely inhibited. Thus, in preferred embodiments the expression of miR-142-5p is completely inhibited or knocked out or knocked down. Appropriate methods would be well known in the art and would include insertion of additional sequences within the endogenous genomic sequence encoding miR-142-5p, for example via lentivirus or other viral vectors, or via well described gene editing techniques such as CRISPR and other techniques such as base editing, zinc finger nucleases or Transcription activator-like effector nucleases (TALENs). Homologous recombination techniques could also be used.

Alternative methods would include inhibiting miR-142-5p expression or reducing or decreasing miR-142-5p levels, e.g. endogenous miR-142-5p levels, in the cytoplasm. Standard methodology can be used to carry out such inhibition, reduction or decreasing. For example, appropriate methods will generally be based on antisense approaches or antisense-like approaches in which synthetic single stranded RNAs can act as miRNA antagonists in order to inhibit the action of or degrade the (endogenous) miRNAs, in this case miR-142-5p. These are also known as antagomirs or anti-miRs.

This approach, i.e. reduced levels, e.g. expression levels of miR-142-5p, preferably the deletion or knockout of miR-142-5p, has been shown to result in inhibition of miR-142-5p, increased expression of PDE3B and reduced expression of cAMP (see the experimental Examples).

The therapeutic methods of this aspect of the invention can be used to treat any cancer, for example blood cancers or solid tumours, in particular solid tumours. The therapeutic methods of this aspect are thus used for cancer immunotherapy. As this aspect of the invention will involve a reduction or inhibition of Treg function, for example reducing the ability of Tregs to suppress Teffs, for example the Tregs may be less able to inhibit proliferation and/or activation of Teffs, cancers which are particularly appropriate for treatment may be those in which Treg suppression of Teffs is particularly dominant or enhanced, for example cancers where large or significant numbers of Tregs are found within and surrounding the tumour, for example Treg infiltrated tumours.

The terms “reduce” or “decrease” (or equivalent terms) as described herein in relation to the level or level of expression of miR-142-5p in a Treg cell (or population of Treg cells) includes any measurable reduction or decrease when compared with an appropriate control. Appropriate controls would readily be identified by a person skilled in the art and would include the level of miR-142-5p in an unmodified Treg cell (or population of unmodified Treg cells), e.g. the level of endogenous expression in a Treg cell, e.g. a Treg which had not been subject to modification, e.g. genetic engineering or modification, to reduce or decrease the level of miR-142-5p. As described elsewhere herein, such unmodified Tregs could be those from a subject with cancer, e.g. a subject to be treated in accordance with this aspect of the present invention, or a comparison could be made with levels of miR-142-5p in Tregs from a population of such subjects, e.g. a comparison could be made with predetermined levels of miR-142-5p. Preferably any such decrease in levels of miR-142-5p will also result in an increase, e.g. a measurable or significant increase in the levels or expression of PDE3B, and/or a reduction (or decrease), e.g. a measurable or significant reduction (or decrease) in intracellular cAMP levels, e.g. when compared to the appropriate control. Preferably the reduction, decrease (or equivalent) will be significant, for example clinically or statistically significant.

T effector (Teff) cells as referred to herein, are a subpopulation of T cells which have a pro-inflammatory role in the immune system. Such cells are also referred to herein as Tconv. Teffs generally express the markers CD4+ or CD8, intermediate levels of CD25 and for example typically express effector cytokines such as interferon-gamma, TNF-alpha, IL-4, IL-5, IL-17 and IL-22. They typically also express lineage defining transcription factors such as T-bet, GATA-3 and RORC.

As set out above, such Tregs (modified Tregs) are useful in the treatment of cancer. Thus, this aspect of the invention further provides such Tregs (modified Tregs) for use in the treatment of cancer.

A yet further aspect of this embodiment of the invention provides a method for preparing (or producing) Tregs suitable for use or for use in the treatment of cancer, said method comprising the following steps:
i) Isolating Tregs from a sample taken from a subject, preferably a blood sample;
ii) Modifying the Tregs, e.g. by genetic modification or genetic engineering, so that the level of miR-142-5p (CAUAAAGUAGAAAGCACUACU, SEQ ID NO:1) or a variant thereof is reduced or decreased; and optionally
iii) in vitro expansion of the Treg cells.

Appropriate methodology for carrying out the above steps is described herein or would be well known to a person skilled in the art. The steps may be carried out in any appropriate order, although preferably the steps are carried out in the order shown. For example, conveniently the expansion step can be carried out after the modification step, in particular where said modification is a stable or inheritable modification. In some embodiments however, for example where said modification is not a stable or inheritable modification, the expansion step could be carried out before the modification step. Preferably all of the steps will be carried out, although in some embodiments, the Tregs for modification may be obtained from a different source or may have already have been isolated in which case an appropriate step would for example involve modifying isolated Tregs or simply modifying Tregs before an optional expansion step.

A yet further aspect of the invention provides a population of Tregs (modified Tregs) produced, obtained or obtainable by the above methods.

Other features and preferred features as described elsewhere herein in connection with the aspects of the invention relating to increasing levels of miR-142-5p in Tregs apply mutatis mutandis to this aspect of the invention relating to reduced or decreased levels of miR-142-5p in Tregs, where appropriate.

As described elsewhere herein, in preferred embodiments of the therapeutic uses of the invention, the therapies are autologous therapies. Thus, in such embodiments the subjects (or patients) treated by the therapy (e.g. AI or cancer subjects, as appropriate) are also the source of the T cells (Tregs) to be used in the therapeutic methods, which are subjected to modification, e.g. genetic engineering or genetic modification, to increase or decrease the levels of miR-142-5p as desired or appropriate.

The term “subject” as used herein includes any mammal, for example humans and any livestock, domestic or laboratory animal. Specific examples include mice, rats, pigs, cats, dogs, sheep, rabbits, cows, horses and monkeys. Preferably, however, the subject is a human subject and thus preferred Tregs are human Tregs. In embodiments relating to therapeutic methods and uses described herein, appropriate subjects are those having, suspected of having, or at risk of having the disease to be treated (e.g. AI or cancer subjects, as appropriate).

Any appropriate route of administration may be used for the Tregs or miR-142-5p molecules. Appropriate routes can conveniently be determined by a person skilled in the art depending on the disease to be treated and the particular formulation of the agent. Typically such agents are administered parenterally, for example by intravenous injection. As described elsewhere herein, the various agents may be provided with additional means of targeting the tissues or organs affected by disease, for example T cells may be targeted by the expression of homing markers such as gut homing markers, or by the presence of antigen targeting moieties, for example by way of CARs.

Appropriate doses of the Tregs (e.g. numbers of Tregs, for example in vitro expanded Tregs), the miR-142-5p molecules and any other agents as described herein, can readily be chosen depending on the disease (or condition) to be treated, the mode of administration and the formulation concerned. Thus, a dosage and administration regime is generally chosen such that the Tregs or miR-142-5p molecules or the other agents administered to the subject in accordance with the present invention can result in the desired therapeutic effects or health benefits (for example the treatment of AI disease or cancer as appropriate, or any other reduction or alleviation of the relevant disease or symptoms of disease). In other words, an appropriate dose is selected so as to be therapeutically effective. Doses can be readily determined by a person skilled in the art by carrying out appropriate experiments and eventually clinical trials.

Also, the indefinite articles “a” and “an” preceding an element or component of the invention are intended to be nonrestrictive regarding the number of instances, i.e., occurrences of the element or component. Therefore “a” or “an” should be read to include one or at least one, and the singular word form of the element or component also includes the plural unless the number or context is obviously meant to be singular.

In addition, where the terms “comprise”, “comprises”, “has” or “having”, or other equivalent terms are used herein, then in some more specific embodiments these terms include the term “consists of” or “consists essentially of”, or other equivalent terms. Lists “consisting of” various components and features as discussed herein can also refer to lists “comprising” the various components and features. Methods comprising certain steps also include, where appropriate, methods consisting of these steps.

The term “decrease” or “reduce” (or equivalent terms) as described herein, e.g. in relation to Tregs, Teffs, expression levels, markers, etc., includes any measurable decrease or reduction when compared with an appropriate control. Appropriate controls would readily be identified by a person skilled in the art and might include non-treated subjects or a level of a particular parameter in the same individual subject (or T cells from that subject) measured at an earlier time point (e.g. comparison with a “baseline” level in that subject). Preferably the decrease or reduction will be significant, for example clinically or statistically significant.

The term “increase” or “enhance” (or equivalent terms) as described herein, e.g. in relation to Tregs, Teffs, expression levels, markers, etc., includes any measurable increase or elevation when compared with an appropriate control. Appropriate controls would readily be identified by a person skilled in the art and might include non-treated subjects or a level of a particular parameter in the same individual subject (or T cells from that subject) measured at an earlier time point (e.g. comparison with a “baseline” level in that subject). Preferably the increase will be significant, for example clinically or statistically significant.

Thus, references herein to significant effects, e.g. significant increases or decreases, preferably refer to statistically significant effects.

Methods of determining the statistical significance of differences between test groups of subjects or differences in levels of a particular marker or parameter are well known and documented in the art. For example herein a reduction/decrease or elevation/increase (or equivalent terms) in level of a particular parameter or a difference between test groups of subjects is generally regarded as statistically significant if a statistical comparison using a significance test shows a probability value of <0.05. Further details are provided in the experimental Examples.

With reference to various sequences as described herein % identity takes its art recognised meaning in which identical sequences have identical nucleic acid residues at the same (or equivalent or corresponding) positions, and may be assessed by any convenient method. However, for determining the degree of homology between sequences, computer programs that make multiple alignments of sequences are useful, for instance Clustal W (Thompson et al., Nucleic Acids Res., 22:4673-4680,1994). Other methods to calculate the percentage identity between two sequences are generally art recognized and include, for example, those described by Carillo and Lipton (SIAM J. Applied Math., 48:1073, 1988).

Generally, computer programs will be employed for such calculations. Programs that compare and align pairs of sequences, like ALIGN (Myers and Miller, CABIOS, 4:11-17, 1988), FASTA (Pearson, Methods in Enzymology, 183:63-98, 1990) and gapped BLAST (Altschul et al., Nucleic Acids Res., 25:3389-3402, 1997) are also useful for this purpose.

By way of providing a reference point, sequences according to the present invention having certain % identity may be determined using the ALIGN program with default parameters (for instance available on Internet at the GENESTREAM network server, IGH, Montpellier, France).

The invention will be further described with reference to the following non-limiting Examples, which also describe some preferred features and embodiments of the invention:

### EXAMPLES

### Example 1

### Results

MicroRNA-142 is Associated with a Super-Enhancer Occupied by FOXP3 in T_{REGS}

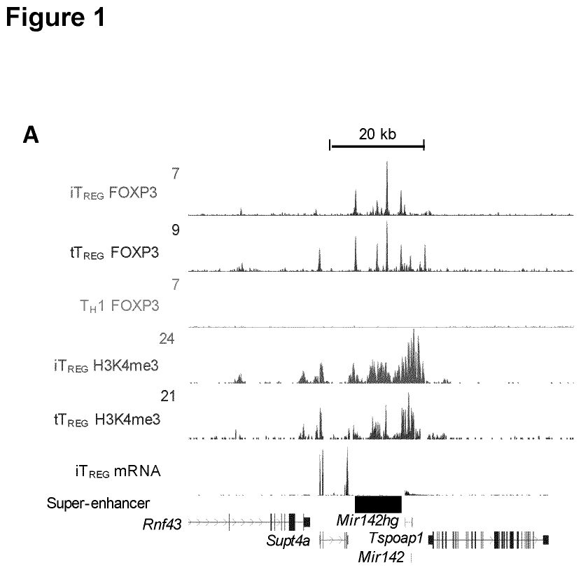

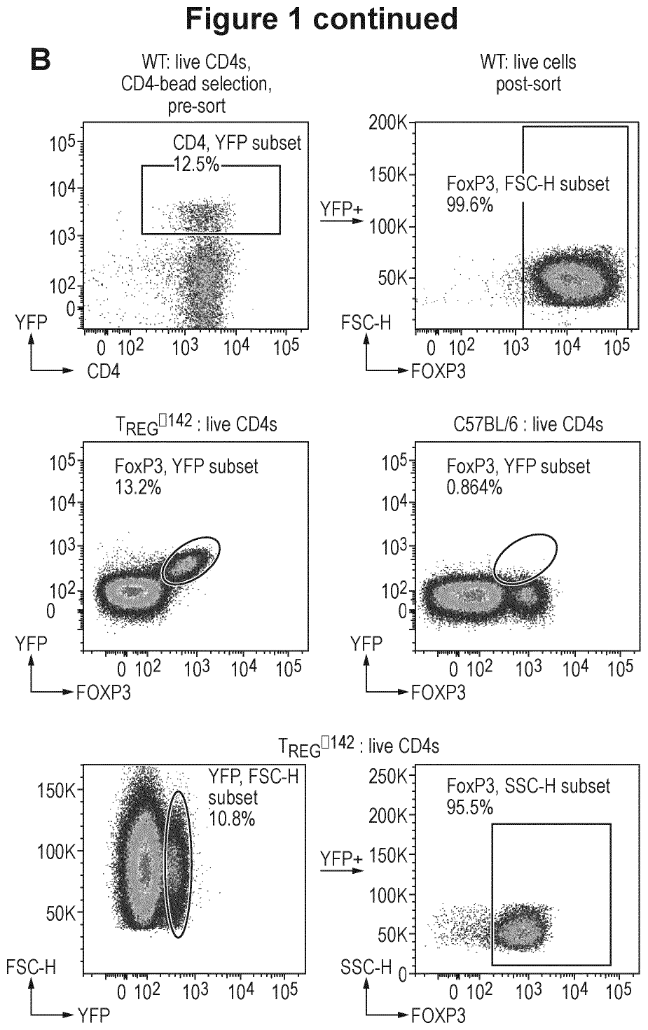

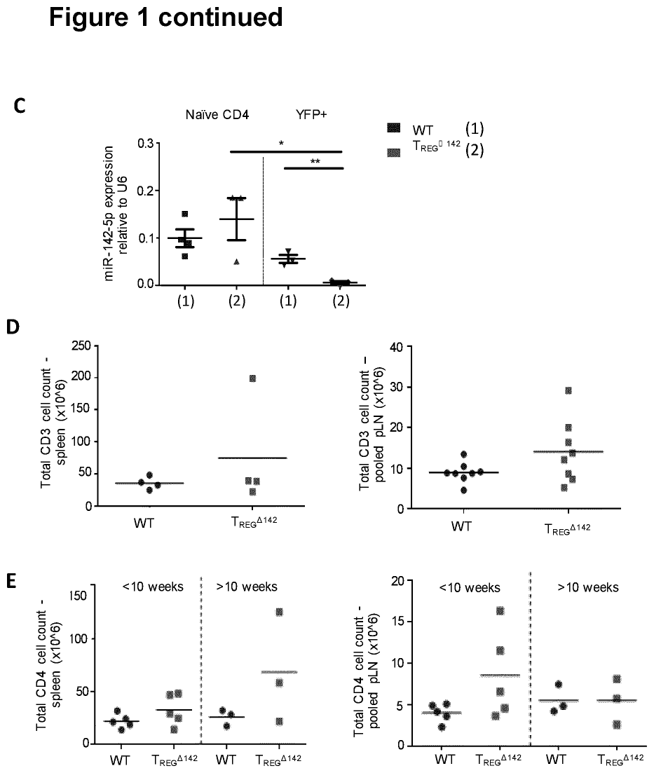

We first sought to identify miRNA genes related to the commitment to, or function of, the CD4⁺ T_{REG} lineage. To address this, we utilized chromatin immunoprecipitation coupled with next-generation sequencing (ChIP-seq) to identify whether any miRNA genes were associated with super-enhancers occupied by FOXP3, the lineage-determining transcription factor (LDTF) of T_{REGS}. Super-enhancers are genomic regions that exhibit particularly high occupancy of LDTF and transcriptional co-activators and tend to be associated with cell type-specific genes (24). The identification of super-enhancers has previously allowed for the definition of key lineage-specific genes critical for controlling T cell identity (24-26). Following our analysis, the only miRNA gene associated with a super-enhancer bound by FOXP3 in T_{REGS} was Mir142, located ˜2 kb upstream of the 5^{th} ranked FOXP3-associated super-enhancer (FIG. 1A, Table 1). FOXP3 bound this locus both in thymically derived T_{REGS} analysed directly ex vivo (tT_{REG}) as well as in T_{REGS} induced in vitro from naïve CD4⁺ T cells activated in the presence of TGF-β and IL-2 (iT_{REG}) (FIG. 1A). The Mir142 locus was also associated with high levels of histone H3 lysine-4 tri-methylation (H3K4me3) in both tT_{REG} and iT_{REG} and was transcriptionally active (FIG. 1A). These data suggested that miR-142 is important for T_{REG} function.

Generation and Validation of T_{REG}^{Δ142} Mice

We next set out to determine whether miR-142 was important for T_{REG} biology or immune tolerance. To do this, we undertook a conditional gene-knockout strategy and generated a T_{REG}-specific miR-142-deficient mouse (FoxP3^{YFP-Cre}×Mir142^{fl/fl}; T_{REG}^{Δ142}). The FoxP3^{YFP-Cre} model allowed for deletion of a target gene in T_{REG} cells only, as previously described (27). Furthermore, because the FoxP3^{YFP-Cre} allele is X-linked, this allowed assessment of the effects of mosaic disruption of miR-142 in female mice that are heterozygous for FoxP3^{YFP-Cre} (FoxP3^{YFP-Cre/WT}×Mir142^{fl/fl}). For clarity, T_{REG}^{Δ142} refers only to male FoxP3^{YFP-Cre}×Mir142^{fl/fl} and homozygous female FoxP3^{YFP-Cre}×Mir142^{fl/fl} mice.

FoxP3 staining of YFP⁺ cells from these mice demonstrated that >95% of FACS-sorted YFP⁺ cells were FoxP3⁺ (FIG. 1B), confirming the validity of YFP as a means of identifying T_{REGS} in this model. Quantitative PCR (qPCR) conducted on material isolated from YFP⁺ cells sorted from T_{REG}^{Δ142} mice confirmed a complete absence of miR-142 transcripts, which were otherwise readily detectable in control cells, validating Cre-mediated deletion of the Mir142 locus under activity of the FOXP3 promoter. (FIG. 1C). Non-T_{REG} T cell populations from these mice demonstrated miR-142 expression levels comparable to controls, demonstrating a lack of detectable background Cre-recombinase activity in this model (FIG. 1C). These mice also did not demonstrate any of the lymphopenic features previously reported in Mir142^{-/-} animals (28), retaining a full spectrum of other T-cell lineages with equivalent levels of miR-142 in naïve CD4⁺ T cells from wild-type (WT; FoxP3^{YFP-Cre}×Mir-142^{+/+}), T_{REG}^{Δ142} and WT YFP⁺ mice (FIG. 1D-E), further confirming T_{REG}-specific deletion of miR-142 in T_{REG}^{Δ142} mice.

T_{REG}^{Δ142} Mice Demonstrate a Defect in T_{REG} Suppressive Function Despite Apparently Normal T_{REG} Lineage Development

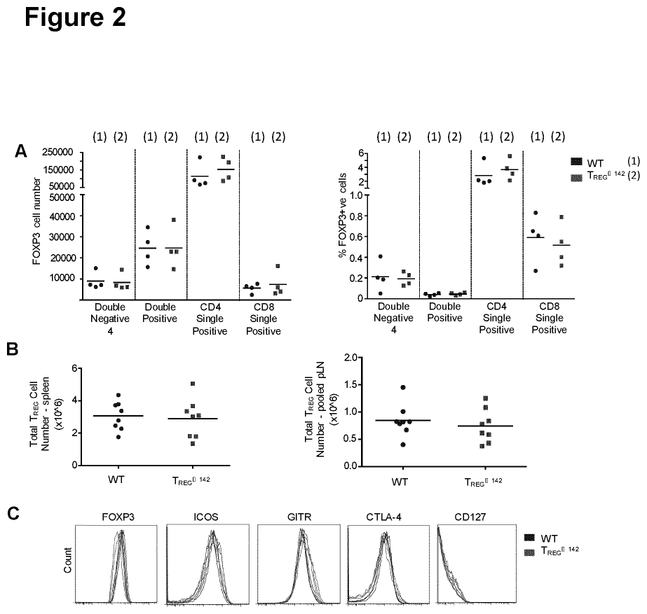

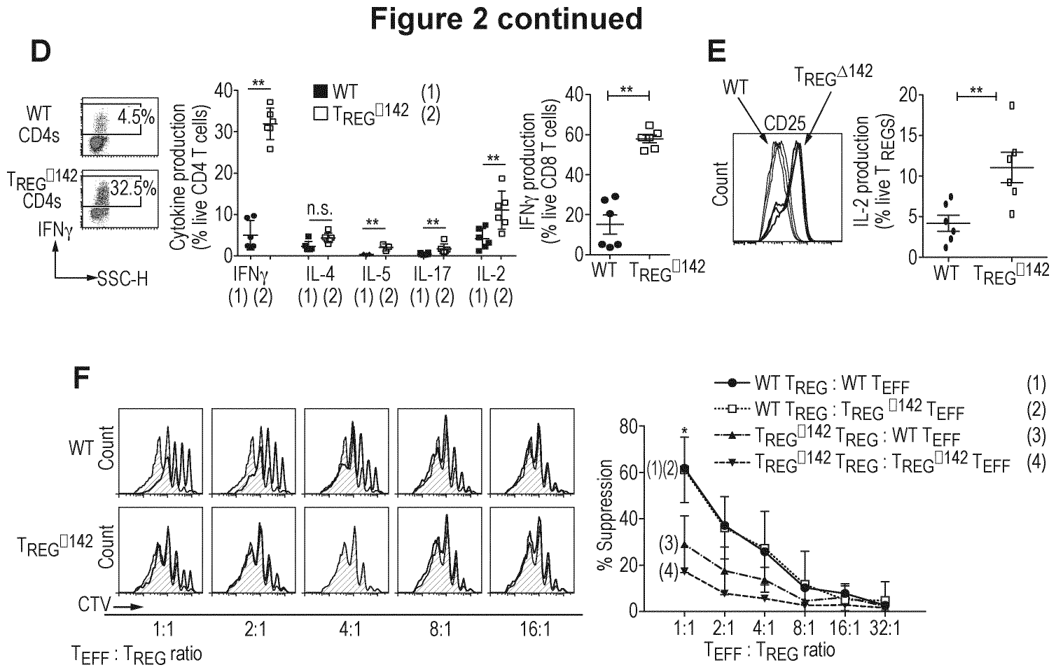

To establish the impact of T_{REG}-specific miR-142 deletion on the development and biological function of T_{REG}^{Δ142} T_{REGS}, as well as the maintenance of peripheral immune tolerance, we examined the T cell compartment of the immune system of these animals in more detail. Immunological phenotyping of T_{REG}^{Δ142} mice at 5-6 weeks of age revealed normal numbers of T_{REGS} in the thymus (FIG. 2A), spleen and peripheral lymph nodes (FIG. 2B). The expression of FOXP3 was unaffected in the absence of miR-142 (FIG. 2C) and the canonical T_{REG} markers ICOS, GITR, CTLA-4 were equivalently expressed between peripheral T_{REG}^{Δ142} and WT T_{REGS}, alongside the low expression of CD127 (IL-7Rα) typical of T_{REGS} (FIG. 2C). Intracytoplasmic cytokine staining of CD4⁺ cells isolated directly ex vivo from T_{REG}^{Δ142} mice demonstrated an increased proportion of cells expressing cytokines, including IFN-γ, IL-2, IL-4, IL-5 and IL-17, versus WT CD4⁺ T cells (FIG. 2D, left), representing unchecked peripheral T_{EFF} responses. In addition, 60% of CD8⁺ T cells from T_{REG}^{Δ142} mice produced IFN-γ, versus <20% of CD8⁺ T cells from WT mice (FIG. 2D, right). T_{REGS} from T_{REG}^{Δ142} mice also demonstrated up-regulated CD25 expression and IL-2 production (FIG. 2E), which may reflect a compensatory increase in T_{REG} activation in the absence of miR-142. T_{REGS} purified from T_{REG}^{Δ142} mice at 5-6 weeks of age completely failed to suppress T_{EFF} proliferation in an in vitro co-culture suppression assay (FIG. 2F), revealing a defect of T_{REG} suppressive function despite apparently normal T_{REG} lineage development and increased T_{REG} activation. These data indicated that following commitment to the T_{REG} lineage, miR-142 is not essential for the maintenance of the peripheral T_{REG} pool.

However, its expression is critical for maintenance of T_{REG} activation state and optimal suppression of cytokine production by T_{EFFS} under steady-state conditions.

The T_{REG}^{Δ142} T_{REG} Suppressive Defect is Cell Intrinsic and Leads to Development of Lethal Multisystem Inflammatory Disease.

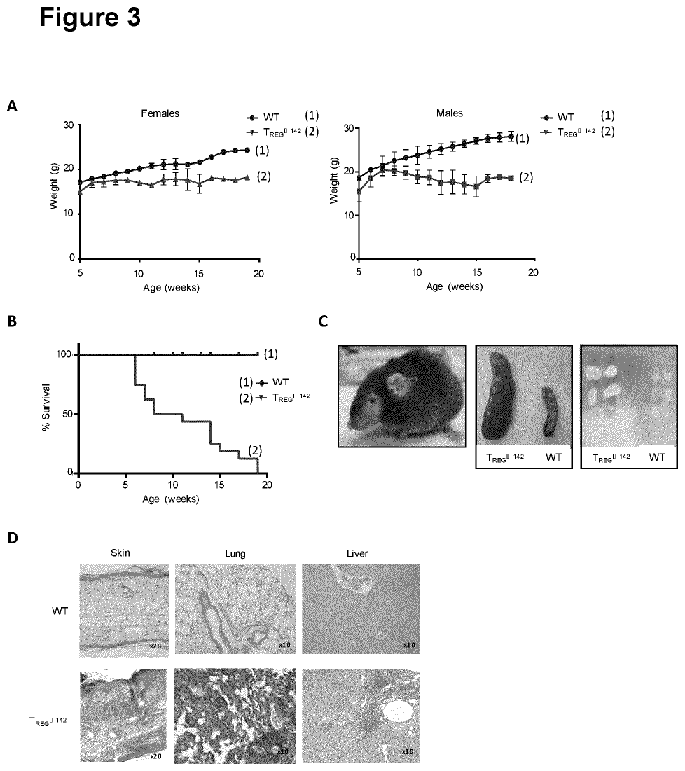

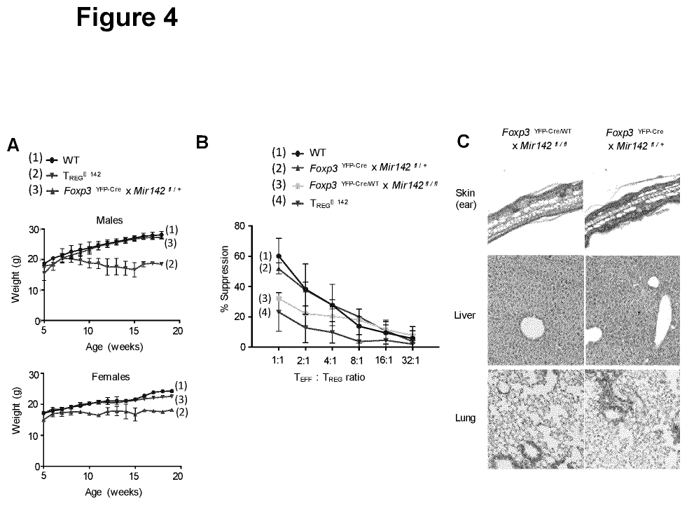

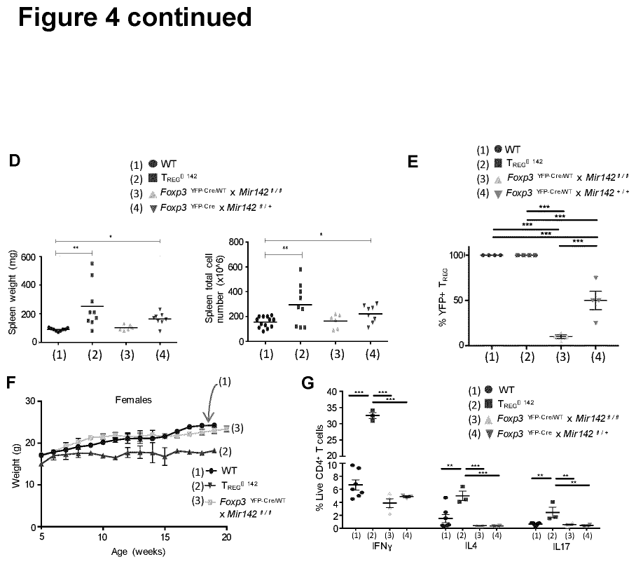

Given the functional suppressive defect displayed by T_{REG}^{Δ142} T_{REGS}, we anticipated that these animals may develop a Scurfy-like phenotype and indeed, this was the case. Starting from 6-8 weeks of age, mice homozygous for the Mir142^{fl/fl} allele, and possessing either one (male) or two (female) FoxP3^{YFP-Cre} alleles, developed a severe multisystem inflammatory disease, characterised by runting, weight loss, and death by 20 weeks (FIG. 3A-B). An early macroscopic feature of the disease phenotype was extensive dermatitis (FIG. 3C). Post-mortem examination was notable for marked lymphadenopathy and splenomegaly (FIG. 3C). Histological examination of the liver, lungs and skin of affected mice also revealed profound lymphohistiocytic infiltration (FIG. 3D), very similar to that seen in the Scurfy mouse, as well as in mice lacking T_{REG}-specific Dicer and Drosha (7, 12-15). In comparison to FoxP3^{YFP-Cre} mice homozygous for the floxed miR-142 allele, male and female FoxP3^{YFP-Cre} mice heterozygous for the floxed allele (Mir142^{fl/+}) did not become terminally ill and remained visually healthy up to at least 12 months of age with no weight loss or overt signs of disease (FIG. 4A). Furthermore, T_{REGS} from these mice retained normal suppressive activity in vitro (FIG. 4B). However, histological examination of the liver, lungs and skin revealed some mild/patchy inflammation (FIG. 4C), accompanied by modest splenomegaly (FIG. 4D). This indicated that while, for the most part, mice haplodeficient for miR-142 expression in T_{REGS} were able to maintain T_{REG} suppressive activity, full peripheral tolerance requires normal homozygous expression of miR-142, highlighting that the precise miR-142 gene dosage is critical to endow full suppressive function.

Since the FoxP3^{YFP-Cre} allele is X-linked, only one of the two FoxP3^{YFP-Cre} alleles is transcriptionally active in any given T_{REG} from a female mouse heterozygous for FoxP3^{YFP-Cre} expression (FoxP3^{YFP-Cre/WT}). Due to this random X-inactivation, ˜50% of T_{REGS} in female FoxP3^{YFP-Cre/WT} mice should be predicted to express YFP (denoting FOXP3-Cre activity). However, while close to 50% of the total T_{REG} pool from FoxP3^{YFP-Cre/WT}×Mir142^{+/+} mice were found to be YFP⁺, in female FoxP3^{YFP-Cre/WT} animals possessing the floxed allele (FoxP3^{YFP-Cre/WT} x Mir142^{fl/fl}), YFP⁺ cells (i.e. those in which the Mir142 locus had been deleted) were found to represent less than 10% of the total T_{REG} pool (FIG. 4E). This observation implied a relative homeostatic defect of miR-142 deficient T_{REGS} in the presence of miR-142-sufficient T_{REGS} in vivo. This conclusion was further supported by the finding that female FoxP3^{YFP-Cre/WT} x Mir142^{fl/fl} mice remained healthy up to at least 24 months of age, with no weight loss, overt disease, or histological evidence of inflammation (FIGS. 4F and 4C). Similarly, cytokine secretion profiles of CD4⁺ T cells from these mice were comparable to littermate control WT mice, demonstrating that the predominance of miR-142-sufficient T_{REGS} maintained immunological tolerance (FIG. 4G) However, in comparison to YFP^{neg} T_{REGS}, YFP⁺ T_{REGS} from FoxP3^{YFP-Cre/WT}×Mir142 . . . animals exhibited reduced in vitro suppressive activity of T_{EFFS} (FIG. 4B), similar to the levels observed in YFP⁺ T_{REGS} from T_{REG}^{Δ142} mice. From this we infer that the miR-142 deficient T_{REG} suppressive defect is cell intrinsic and directly attributable to cell-specific loss of miR-142 expression.

Pde3b is a Direct Target of miR-142-5p in T_{REGS}

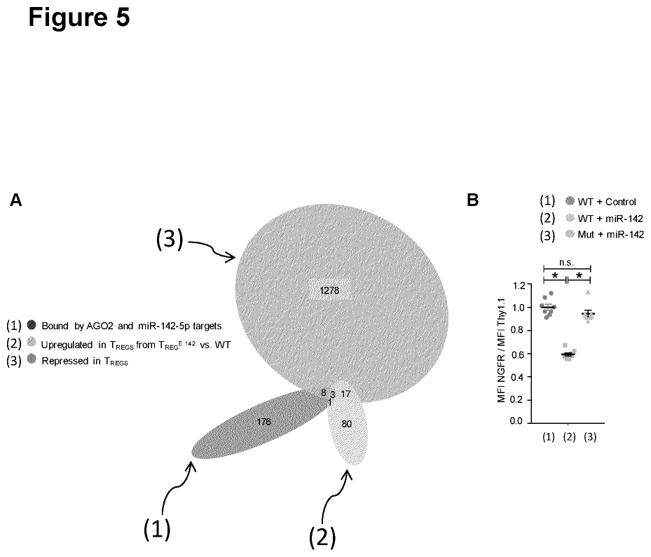

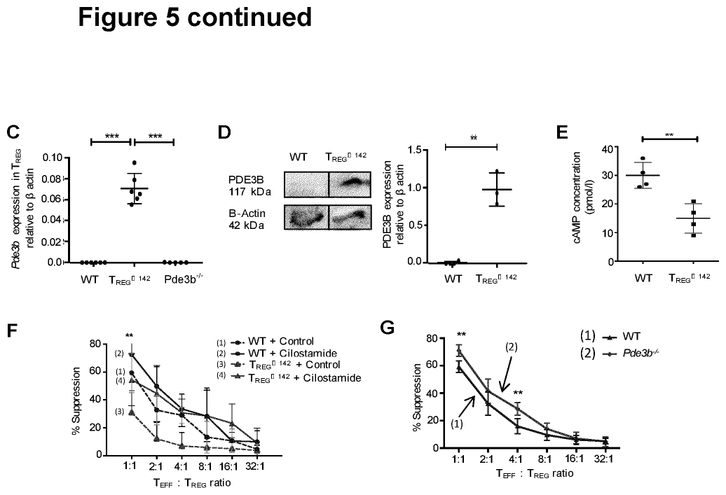

To understand the mechanism underlying the phenotype associated with T_{REG}-specific miR-142 deletion, we next attempted to identify genes directly regulated by miR-142 in T_{REGS}. Argonaut 2 (AGO2) binds mature miRNAs as part of miRISC, which directs translational inhibition and degradation of target mRNAs (2). Therefore, to begin our target identification screen, we first identified mRNAs bound by AGO2 at predicted miR-142 target sites in activated CD4⁺ cells, utilizing publicly available AGO2 high-throughput sequencing of RNA isolated by crosslinking immunoprecipitation (HITS-CLIP) data (29). We further reasoned that T_{REG} miR-142 target genes should be upregulated in T_{REGS} in the absence of miR-142 and downregulated in WT T_{REG} vs T_{EFFS}. Thus, we required that candidate miR-142 target genes were upregulated (>2-fold, p<0.05) in T_{REG}^{Δ142} T_{REG} vs WT T_{REG} and downregulated (>2-fold, p<0.05) in WT T_{REGS} vs T_{EFFS} (30). As miR-142-3p is minimally expressed in T_{REGS} compared with miR-142-5p (18), we reasoned that miR-142-5p was likely to be the main active mature miR-142 species in T_{REGS}. Application of these stringent criteria identified three candidate miR-142-5p target genes; Pde3b, Epas1 and Igf2bp3 (FIG. 5A). To confirm whether these genes were directly targeted by miR-142 as predicted, we utilized a flow cytometry-based reporter assay. The inclusion of the Pde3b 3′UTR region containing the predicted binding site in the reporter construct led to robust repression of reporter gene expression in the presence of miR-142 (FIG. 5B). This repression was completely reversed by mutation of the seed sequence at the predicted target site (data not shown). In contrast, the repression was modest for the regions containing the predicted binding sites in the Epas1 and Igf2bp3 3′UTRs and was not relieved by mutating the seed sequences in either case (data not shown). Therefore, we concluded that Pde3b is a direct target of miR-142-5p and that the predicted miR-142-5p binding sites in the 3′UTRs of Epas1 and Igf2bp3 are not truly functional.

PDE3B regulates the intracellular concentration of cAMP and is critical for T_{REG} function (23,25). We therefore sought to determine whether the critical requirement of miR-142 for T_{REG} suppressive function was associated with the direct repression of Pde3b by miR-142-5p. Supporting the hypothesis that miR-142-5p directly targets Pde3b in T_{REGS}, qPCR confirmed significant overexpression of Pde3b in T_{REG}^{Δ142} T_{REGS} compared to controls observed by RNA-seq (FIG. 5C). However, Pde3b transcript levels expressed by non-T_{REG} T cell populations in T_{REG}^{Δ142} and WT mice were not significantly different to one another (data not shown), further validating the specificity of deletion of miR-142 in T_{REG}^{Δ142} T_{REGS}. In agreement with previous reports, we found that PDE3B protein was not detectable in WT resting T_{REGS} (data not shown) (23). However, PDE3B protein was clearly present in T_{REG}^{Δ142} T_{REGS}, as detected by Western blot (FIG. 5D), congruent with findings of elevated Pde3b levels by qPCR. Consistent with increased PDE3B protein, intracellular cAMP levels in T_{REG}^{Δ142} T_{REG} lysate were lower compared with WT T_{REG} control lysate (FIG. 5E), indicating that not only was there more PDE3B protein present in T_{REG}^{Δ142} T_{EEGS}, but that the enzyme was also active. Importantly, the miR-142-5p target site in the Pde3b 3′UTR is highly conserved among mammals (Table 2) supporting the likely translation of these findings in mice to human biology. Combined, these results show that Pde3b transcript levels are directly regulated by miR-142-5p in T_{REGS}, leading to optimal T_{REG} suppressive capacity through the maintenance of intracellular cAMP.

Disruption of PDE3B Activity Through Pharmacological Inhibition or Genetic Ablation Restores T_{REG}^{Δ142} T_{REG} suppressive function and prevents lethal autoimmune disease

If the suppressive capacity of T_{REGS} from T_{REG}^{Δ142} mice was impaired due to elevated expression of Pde3b in the absence of miR-142, inhibition of PDE3B would be predicted to reverse this effect. Cilostamide is a competitive inhibitor of PDE3A and PDE3B, but of the two isoforms, only PDE3B is present in T cells (22,23). We found that pre-treatment of T_{REGS} from 5-6 week old WT and T_{REG}^{Δ142} mice with cilostamide (10 μM) prior to co-culture with untreated T_{EFFS} in the absence of cilostamide restored the suppressive function of T_{REG}^{Δ142} T_{REGS} (FIG. 5F). No rescue of suppressive function was observed when T_{EFFS} were pre-treated with cilostamide (not shown). T_{REGS} from Pde3b-deficient mice demonstrate marginal augmentation of ex vivo suppressive capacity when compared with WT T_{REGS}, similar to that seen in WT T_{REGS} treated with cilostamide in vitro (FIG. 5F-G). These data suggest that PDE3B may be expressed following TCR ligation in WT T_{REGS}, or that perhaps a low level of transient PDE3B expression may exist, consistent with previous reports (23). Importantly, whilst we observed some reduction of T_{REG} viability in the absence of miR-142 during in vitro culture, mirroring the in vivo loss of competitive fitness in the presence of WT T_{REGS}, the recovery of suppressive function following pharmacological inhibition of PDE3B was completely independent of this defect (data not shown). Thus, our data indicate that the defect in T_{REG} function in the absence of miR-142 is independent of any observed homeostatic defect and is rescued by T_{REGS}-selective PDE3B inhibition.

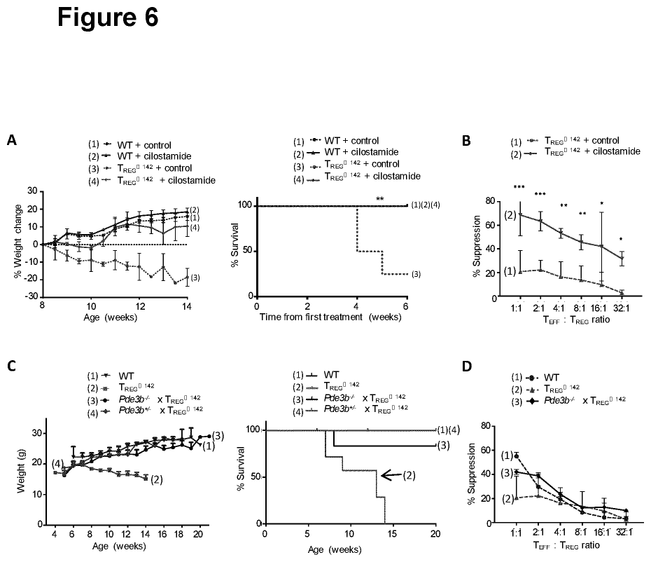

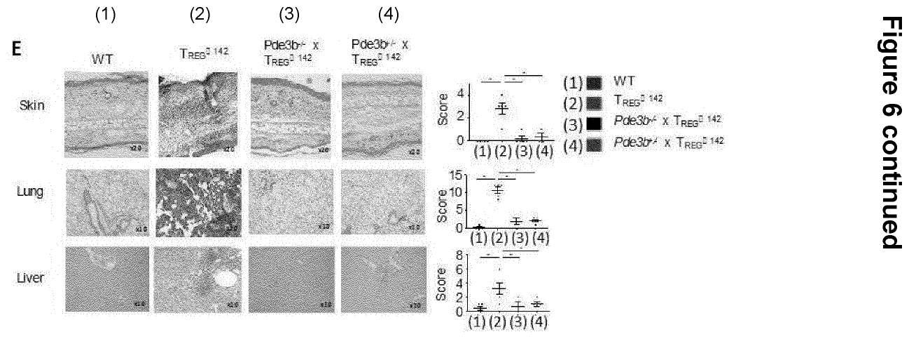

We next sought to determine the relevance of this pathway in vivo. Remarkably, treatment of T_{REG}^{Δ142} mice with cilostamide from 8 weeks of age prevented lethal autoimmune disease (FIG. 6A), which was correlated with fully restored suppressive activity of miR-142-deficient T_{REGS} (FIG. 6B). To further validate that the defect observed in miR-142 deficient T_{REGS} was a direct function of increased PDE3B levels, we generated a Pde3b-deficient, T_{REG}-specific miR-142-deficient mouse (Pde3b^{-/-}×FoxP3^{YFP-Cre}×Mir142^{fl/fl}; Pde3b^{-/-}×T_{REG}^{Δ142}). We found that these mice (in addition to littermates heterozygous for Pde3b germline deletion) remained healthy up to >20 weeks of age, with no weight loss, dermatitis or overt disease and exhibited restored ex vivo T_{REG} suppressive function (FIG. 6C-D). Histological examination of the liver, lung and skin showed evidence of only mild, patchy inflammatory infiltrate (FIG. 6E). To exclude the possibility that this was due to impaired immunity in Pde3b-deficient mice, we examined their T cell effector function. Pde3b^{-/-} mice had normal T cell numbers and effector function when compared with WT T_{EFFS}, as previously reported (23) (data not shown).

We therefore conclude that miR-142-5p represses Pde3b expression in T_{REGS} and that this is essential for T_{REG} suppressive function. In the absence of this critical molecular pathway, the mechanisms governing peripheral immune tolerance are compromised, resulting in a systemic lethal auto-immune syndrome.

### Discussion

The restriction of self-reactive peripheral T_{EFF} responses by T_{REGS} is a core tenet of the mechanisms underlying peripheral tolerance, preventing development of autoimmunity. In the work presented here, we have demonstrated that miR-142 plays a critical, cell-autonomous role in facilitation of this T_{REG} suppressive activity and by virtue of this, successful orchestration of peripheral tolerance. By applying stringent target identification criteria and by validating these targets experimentally, the phosphodiesterase Pde3b was revealed to be a direct target of the miR-142 isoform miR-142-5p in T_{REGS}. These data support the conclusion that under WT conditions, direct repression of Pde3b by miR-142-5p is a key determinant of T_{REG} function and peripheral immune tolerance.

The work presented in this study contributes significantly to the understanding of the role of miRNAs in T_{REG} biology. Previously, it has been shown that T_{REGS} display a distinct miRNA expression profile when compared with conventional CD4⁺ T_{EFF} (31), suggesting a role for specific miRNAs in the different functions of these cell types. T_{REG}-specific miR-142 deletion did not appear to impact upon T_{REG} lineage development in our study. However, iT_{REG} development has been shown to be positively regulated by miRNAs including miR-15b/16 (32), miR-99a (33), miR-126 (34) and miR-150, which target and suppress key components of the PI3K/AKT/MTOR signaling pathway, favoring T_{REG} induction over T_{EFF} generation (35). Likewise, both iT_{REG} and tT_{REG} development is augmented by suppression of cytokine signaling 1 (SOCS1) through miR-155 (36). In contrast, other miRNAs such as miR-17 (37) and miR-100 (38) can play inhibitory roles on T_{REG} development via suppression of core components of the TGF-β signaling pathway, including TGFβRII and SMAD2/3, or through direct suppression or destabilization of Foxp3 mRNA, as has been shown for miR-10a (39), miR-15a/16 (40), miR-24 (41), miR-31 (42), miR-125a (43), miR-146a (44) and miR-210 (41). We revealed that miR-142-5p exerts its critical function in T_{REGS} via facilitation of T_{REG}-suppression of T_{EFFS}. At the mechanistic level, this is achieved via maintenance of high T_{REG} intracellular cAMP concentration, which is essential for subsequent transfer of cAMP to T_{EFFS}, suppressing their activation (21). In addition to transfer of cAMP, T_{REGS} also suppress T_{EFFS} through CTLA4, limiting T_{EFF} positive co-stimulatory signaling through CD28 interactions with CD80/86 on antigen presenting cells. CTLA4 is reported to be targeted by both miR-15a/16 (40) and miR-145 (41) in T_{REGS}; however these interactions limit CTLA4 expression and reduce T_{REG} suppressive function. Therefore, to our knowledge, this is the first report of a miRNA playing a direct, cell-intrinsic positive role in augmenting T_{REG} suppressive activity.

Interestingly, while our study clearly demonstrates the critical requirement of miR-142-5p for maintenance of high intracellular concentration of cAMP in T_{REGS}, miR-142-3p has previously been reported to restrict cAMP generation in CD4⁺ T cells through targeted suppression of adenylyl cyclase 9 (AC9) (45). Adenylyl cyclases are critically required for cAMP generation and in their report, Huang et al., showed that T_{EFFS} maintain high levels of miR-142-3p in order to restrict AC9 expression, thus limiting endogenous cAMP production, which would otherwise inhibit their cellular activation. However, the authors reported that miR-142-3p expression is minimized in T_{REGS} in order to support AC9 expression, facilitating cAMP generation such that T_{REGS} may carry out suppression of T_{EFFS}. This is in line with other reports detailing miR-142-5p as the predominant miR-142 isoform found in T_{REGS} (17). Our findings together with these data support a model whereby differential expression of miR-142 isoforms maintains T_{REG} cAMP intracellular concentration. The synthesis of cAMP (low miR-142-3p, AC9 increased) as well as the inhibition of its hydrolysis to AMP (high miR-142-5p, PDE3B inhibited) may therefore represent parallel components of the same molecular goal.

Previously it has been proposed that FOXP3 maintains the lineage stability and homeostasis of T_{REGS}, in part by binding to and repressing Pde3b transcription directly, in order to maintain high levels of intracellular cAMP (23,46). However, our data clearly demonstrate that in the absence of miR-142-5p, FOXP3 mediated repression of Pde3b is not sufficient to prevent significant upregulation and activity of PDE3B, leading to reduced intracellular cAMP and the breakdown of peripheral tolerance and lethality. Therefore, FOXP3 and miR-142-5p work in concert to maintain Pde3b repression, consistent with the established role of miRNAs in reinforcing transcriptional programs and conferring robustness to biological processes (47).

The worldwide surge in the incidence and prevalence of autoimmune disease over the last 30 years (including in younger people) is associated with a marked socio-economic burden and has generated a global multi-billion-dollar treatment market (48). Our findings represent a significant step forward in the understanding of the mechanisms governing peripheral immune tolerance and suggest that modulation of this novel pathway can provide a clinically tractable, new route for augmenting protective immune responses. Modulation of this pathway, either through PDE3B inhibition or via exogenous manipulation of miR-142-5p expression levels or treatment with miRNA mimics therefore represent viable treatment options, e.g. for autoimmune diseases. A number of PDE3 inhibitors are already widely used as therapeutic agents, for example Cilostazol in the treatment of intermittent claudication (49, 50). With over 100 clinical trials registered on ClinicalTrials.gov for Cilostazol alone, the pharmacodynamic, tolerability and safety profiles of these drugs have been under careful evaluation for a number of years. However, none of these trials is focused on assessing the potential therapeutic applications of PDE3 inhibitors in the treatment of autoimmune disease. In light of our findings, modulation of PDE3B activity in T_{REGS} may help to re-establish mechanisms of tolerance in patients suffering from autoimmune disease.

In addition to the relevance of our findings for treatment of autoimmune disease, identification of miR-142-5p as a critical metabolic regulator of T_{REG} suppressive function also has direct relevance to cancer immunotherapy, particularly of solid tumors. T_{REGS} are enriched in cancer patients, particularly within and surrounding the tumor (51,52). Furthermore, the number of T_{REG} within tumors is frequently associated with poor clinical outcome for patients (53-56). The reasons for this are incompletely understood but appear related to a tumor microenvironment rich in TGF-β (57), IL-10 (58) and adenosine (59) which promote T_{REG} development, survival and activity, resulting in enhanced suppression of tumor infiltrating T_{EFFS}. Alongside other tactics employed by tumor cells, such as elevated expression of PD-L1 that promotes further suppression of anti-tumor T_{EFF} responses through inhibitory interactions with PD-1 expressed by activated T_{EFFS} (60), tumor-resident T_{REGS} act to abolish anti-tumor T cell immunity through enhanced peripheral tolerance to, or ‘immunological ignorance’ of, the tumor itself. Thus, the ability to manipulate (and preferably reduce) the suppressor function of T_{REGS} on T_{EFFS} by manipulating the levels (in particular by reducing the levels) of miR-142-5p in T_{REGS} has clear therapeutic potential.

In summary, our results indicate that a critical function of miR-142-5p in T_{REGS} is to facilitate T_{EFF} suppression via repression of PDE3B, leading to reduction of intracellular cAMP turnover. Therefore, it is believed that this mechanism is an attractive and novel avenue for T_{REG} targeted immunotherapies of autoimmune disease, solid tumors and for augmenting the response to pathogens.

### Materials and Methods

Animals. Mir142^{fl/fl} mice were generated by homologous recombination in 129S mouse embryonic stem cells using a targeted vector containing both FRT and loxP sites flanking the Mir142 locus, and a neomycin resistance cassette, to enable constitutive and conditional miR-142-deficient generation (performed by Genoway, Lyon, France). Chimeric offspring were bred with C57BL/6J-Flp deleter mice to generate conditional lines, which were subsequently fully back-crossed onto a C57BL/6 background. Appropriate control mice were utilized in all experiments, with age and sex-matched littermate FoxP3^{YFP-Cre}×Mir142^{+/+} mice (WT) as controls for the FoxP3^{YFP-Cre}×Mir142^{fl/fl} (T_{REG}^{Δ142}) line. Pde3b^{-/-} mice were a kind gift from Prof. Vincent Manganiello (National Institute of Health, Bethesda, Md., USA) and generated as previously published (63). Chimeric offspring were bred with C57BL/6 mice to generate heterozygous lines, then bred with FoxP3^{YFP-Cre}×Mir142^{fl/fl} and FoxP3^{YFP-Cre}×Mir142^{+/+} mice to generate appropriate littermate controls to utilize in all experiments. The mice were housed in specific pathogen-free conditions, and all experiments were performed according to King's College London and national guidelines, under a UK Home Office Project License (PPL:70/7869 to September 2018; P9720273E from September 2018).

Flow Cytometry and Intracellular Cytokine Staining. Single cell suspensions were prepared from spleen and peripheral lymph nodes by tissue disruption and filtration. Following red cell lysis of splenocyte suspensions, an aliquot of 5×10⁶ splenocytes was stimulated with phorbol 12-myristate 13-acetate (PMA) at 1 ng/ml (Sigma) and lonomycin at 1 μg/ml (Sigma) for 4 hours at 37° C., 5% CO₂, with the addition of Monensin at 2 μM concentration (Sigma) for the last 2 hours. Stimulated and unstimulated samples were then Fc blocked and surface stained with fluorochrome-conjugated anti-mouse antibodies to Live/Dead (Life Technologies), and combinations of CD45, CD3, CD4, CD8, CD25, CD44, CD62L, ICOS, GITR, CXCR3 and CD127 (eBioscience). A proportion of cells, including those stimulated with PMA and ionomycin, were fixed and permeabilised using a mouse Intracellular Staining kit (eBioscience) as per the protocol, and intracellular stains were then applied with fluorochrome-labelled anti-mouse antibodies to FoxP3, T-bet, CTLA-4, IFNγ and IL-17 (eBioscience). Appropriate single stain controls were utilised for all fluorochromes. Cells were acquired on a Fortessa machine (BD Biosciences) and analysed using FlowJo software (TreeStar).

For thymus flow cytometric analyses, thymocytes were harvested from 5-6 week old mice, Fc blocked and surface stained with fluorochrome-conjugated anti-mouse antibodies reporting Live/Dead (Life Technologies). Half the cells were stained with a general panel consisting of anti-mouse antibodies to CD24, CD25, CD5, TCRβ, CD4 and CD8 (eBioscience), and the other half were stained with a double-negative panel consisting of anti-mouse antibodies to CD44, CD25, and primary biotinylated antibodies to CD3, CD4, CD8, CD19, TCRγδ, CD11b, CD11c, Ly6G, NK1.1 and Ter119, with a subsequent secondary, fluorochrome-conjugated streptavidin step. All cells were fixed and permeabilised (as before), stained for Foxp3, and acquired and analysed as before. For cells stained with the double-negative panel, dead cells and streptavidin-positive cells were excluded and the remaining cells were gated into successive double-negative populations by CD44 and CD25 (DN1 CD44+ CD25−, DN2 CD44+ CD25+, DN3 CD44− CD25+, DN4 CD44− CD25−).

In vitro Suppression Assay. CD4⁺ T cells were isolated from pooled peripheral lymph nodes and spleens of 5-6 week old mice using CD4 microbeads (Miltenyi Biotec). Cells were labeled with fluorochrome-conjugated anti-mouse antibodies to CD4, CD62L, CD44 and CD25 (eBioscience) and sorted using a BD FACSAria II flow cytometric cell sorter (BD Biosciences) to >95% purity for YFP⁺ CD4⁺ cells (T_{REGS}) and naïve (CD25−, CD62L+, CD44−) CD4+ T cells (T_{EFFS}). An aliquot of the sorted T_{REG} population was stained with Live-Dead and anti-CD25 antibodies, fixed and permeabilised, then stained for FoxP3 and acquired on a flow cytometer (as before) to confirm purity. The T_{EFFS} were labeled with 10 μM Cell Trace Violet (Life Technologies) according to the manufacturer's instructions, washed and then cultured in a 96-well U-bottom plate alone or with T_{REGS} at ratios ranging from (T_{EFF}:T_{REG}) 1:1 to 32:1, in triplicate, in the presence of anti-CD3 and anti-CD28 Dynabeads (Life Technologies) at a bead:cell ratio of 2:1, and RPMI 1640 cell culture medium (Gibco, Life Technologies) supplemented with 10% fetal calf serum, 50 μM 2-Mercapto-ethanol, 2 μM L-glutamine, pyruvate, HEPES, non-essential amino acids and antibiotics at 37° C. 5% CO₂. All 4 possible combinations of T_{REGS} and T_{EFFS} from each group were utilised. After 72 hours in culture, the cells were stained with fluorochrome-conjugated anti-mouse antibodies to Live/Dead (Life Technologies) and then proliferation of the T_{EFFS} was assessed by flow cytometry based on Cell Trace Violet dilution (excluding YFP+ and dead cells). The numbers of non-proliferating cells (events in the first peak) and precursors of proliferating cells were calculated using standard formulae. Percentage suppression (S) of proliferation was calculated using the formula:

S=100−([c/d]×100)

where c is the percentage of proliferating precursors in the presence of T_{REGS} and d is the percentage of proliferating precursors in the absence of T_{REGS}.

Histology of tissue samples. Mice were sacrificed between 6 and 20 weeks of age with age- and sex-matched WT controls. Samples of liver, lung and ear skin were fixed in 10% neutral buffered formalin for 48 hours before paraffin-embedding, sectioning, and staining with Heamatoxylin & Eosin (Sigma). Sections were scored blinded using histological scoring systems previously published for dermal inflammation (64) and lung/liver injury (65). Microscopy was performed with an Olympus BX51 microscope.

Measurement of Intracellular cAMP. T_{REGS} were isolated by flow cytometric cell sorting as above and washed three times with cold PBS. They were then lysed and the lysates used to measure cAMP using a Parameter cAMP assay kit according to the manufacturer's instructions (R&D Systems).

In vitro Cilostamide treatment. T_{REGS} were isolated by flow cytometric cell sorting as above. They were then cultured in the presence of 10 μM cilostamide (Sigma Aldrich), or equivalent PBS/DMSO control for 48 hours before being washed twice, counted and used in an in vitro co-culture suppression assay, as above. Pre-treatment of T_{REGS} with cilostamide prior to co-culture, as opposed to treatment during the assay was employed to prevent inadvertent treatment of T_{EFFS} as well as T_{REGS}, excluding an effect of cilostamide on non-T_{REG} populations in the suppression co-culture assay.

In vivo cilostamide treatment. Littermate T_{REG}^{Δ142} and WT mice were treated from 8 weeks of age with alternate day intra-peritoneal injections of either 6.4 mg/kg cilostamide (Sigma) in PBS 10% DMSO, or a control solution of PBS 10% DMSO. The mice were weighed every other day and monitored for signs of disease. A proportion of mice were sacrificed after 4 weeks of treatment and T_{REGS} were isolated directly ex vivo for use in an in vitro co-culture suppression assay (as before). A proportion of mice were kept alive for as long as possible (either until they lost more than 15% of their body weight and had to be euthanised, or until the end of the experiment).

RNA extraction and RT-qPCR. Total RNA was extracted using Trisure (Bioline) according to the manufacturer's instructions. MiR-142-5p RT-qPCR, cDNA reverse transcription and RT-qPCR were performed according to the manufacturer's instructions using TaqMan assays (Applied Biosystems), with U6 small nuclear RNA or miR-191-5p endogenous controls. For Pde3b RT-qPCR, cDNA was prepared according to the manufacturer's instructions using the Revertaid cDNA kit (Thermo Fisher), and qPCR was performed using the Maxima Probe/ROX qPCR master mix (Thermo Fisher), with a specific primer/probe set for Pde3b (Life Technologies) and beta-actin as an endogenous control. All qPCR reactions were analysed using an ABI Prism 7900HT real-time PCR instrument (Applied Biosystems). Results were expressed relative to U6 or beta-actin using the 2^{×ΔCt} method.

FoxP3 ChIP-seq in mouse T_{REGS}. CD4⁺ T cells from spleens and lymph nodes of 4- to 10-week old C57BL/6 mice were purified by CD4 positive selection (Miltenyi Biotec) followed by sorting of naive CD4⁺CD25⁻CD62L^{high}CD44^{low} cells using a FACSAria II (BD Biosciences). Cells were activated by plate-bound anti-CD3 and anti-CD28 (both 10 μg/ml; clones 145-2C11 and 37.51, respectively; Bio X Cell). T_{REG} cells were generated by culturing in recombinant human TGF-β1 (33 ng/ml) and IL-2 (20 ng/ml; R&D Systems) for 7 days. Th1 cells were polarised as previously described (26). ChIP for FoxP3 was performed as described (26) using a mix of two antibodies; Santa Cruz sc-31738 and eBioscience FJK-16. Libraries were constructed and sequenced as previously described (26).

Previously published sequencing data. The following datasets were downloaded from GEO:

Mouse nT_{REG} FoxP3 ChIP-seq: GSM999179 and GSM999181 (input) (66).
Mouse iT_{REG} H3K4me3 ChIP-seq: GSM362005 (67).
Mouse nT_{REG} H3K4me3 ChIP-seq: GSM362007 (67).
Mouse iT_{REG mRNA-seq}: GSM1480828 (25).

ChIP-seq and RNA-seq data analysis. Reads were filtered to remove adapters using fastq-mcf, and for quality using seqkt. ChIP-seq reads were aligned to mm9 with Bowtie2 (default settings). FOXP3 ChIP-seq data were filtered for satellites and blacklisted regions (26) and super-enhancers identified with the ROSE algorithm using the default settings (68). RNA-seq data were aligned with TopHat to mm9 (default settings) and converted to bigwig format as described (26).

ChIP-seq data are available at Gene Expression Omnibus (GEO): http://www.ncbi.nlm.nih.gov/geo/query/acc.cgi?token=yjinkewgipuptsp&acc=GSE72279

Microarrays and target prediction algorithms. Total RNA was extracted using TRIsure (Bioline) as before. Complementary DNA (cDNA) was prepared using the Ovation Pico WTA kit (NuGEN Technologies) and hybridized to Affymetrix Mouse Gene ST 2.0 microarrays by the King's College London Genomics Centre facility. All cell populations used for the microarray analysis were generated in duplicates and individually processed. Raw data were processed with the robust multi-array average RMA algorithm (69) for probe-level normalization and differential expression was estimated using the limma package in Bioconductor (70). Data from the arrays (GEO accession number GSE122881) were compared with two public domain datasets; DIANA microT-predicted miR-142-5p target sites within CLIP-defined Ago2 binding sites in activated CD4⁺ T cells (29) and genes down-regulated ≥2-fold at p=0.05 in T_{REGS} vs T_{EFFS} cells (30). Published microarray data was downloaded from GEO under the accession number GSE14350 and analyzed as described above.

Development of flow cytometry-based miRNA-target reporter gene assay. The direct interaction of a miRNA and a putative target is commonly confirmed by using a luciferase reporter assay in which the 3′ UTR of the gene tested is cloned downstream of a luciferase reporter gene. Using such an assay we found that expression of miR-142 led to down-regulation of the Renilla firefly reporter activity independently of the presence of the Pde3b 3′ UTR. These findings are consistent with a previous report (71), which identified several miR-142 binding sites in the Renilla luciferase cDNA. Therefore, we instead generated a new flow-cytometry based reporter system that contains the truncated human low affinity nerve growth factor receptor (NGFR, also called CD271) gene as a read-out for miRNA-target interaction. As an internal transfection control, the vector expresses the cell surface marker Thy1.1 (also called CD90.1) under control of a separate promoter. Both expression cassettes were separated by a synthetic poly(A) signal and transcriptional pause element to stop read-through of the NGFR transcript.

The portion of NGFR encoding the intracellular domain was PCR amplified and inserted into BgIII+EcoRI sites of pcDNA3 (Invitrogen). A second expression cassette consisting of the Thy1.1 cell surface marker under the control of the phosphoglycerate kinase 1 promoter was inserted into NotI+SalI sites of pMY-IRES-EGFP (Cell Biolabs) from which the IRES-EGFP element had been removed. To avoid interference in expression between the two reporter genes a synthetic poly(A) signal/transcriptional pause region was amplified by PCR using pGL4.13 (Promega) as a template, digested with XhoI/EagI and inserted upstream of the PGK promoter into XhoI and NotI sites. A multiple cloning site consisting of restriction sites for EcoRI-NheI-SacII/NotI-XhoI was generated by annealing two synthesized oligonucleotides, which was ligated into EcoRI+XhoI sites. From this vector the MCS-poly(A)-PGK-Thy1.1 cassette was excised with EcoRI+SalI and inserted into EcoRI+XhoI sites of pcDNA-tNGFR resulting in pcDNA-tNGFR-poly(A)-PGK-Thy1.1. A 207 bp fragment of the Pde3b 3′UTR (base position 1448 to 1654) containing a predicted miR-142-5p target site was PCR amplified from cDNA and inserted into NotI+XhoI sites of pcDNA-tNGFR-poly(A)-PGK-Thy1.1. Reporter vectors contained position 1-959 of the Igf2bp3 3UTR and position 2253-2258 of the Epas1 3′UTR. A mutated reporter construct was generated through substitution of five bases in the seed sequence (base position 1566 to 1570) by overlap PCR.

miRNA-target reporter gene assay. HEK293T cells were plated into 12-well plates at 7.3×10⁴ cells/well, 24 hours before transfection. 3.28 ng pcDNA-tNGFR-poly(A)-PGK-Thy1.1 reporter plasmid containing either the wild-type or mutated Pde3b 3′UTR and 1.17 μg pMY-miR-142-IRES-PAC or pMY-IRES-PAC control plasmid were co-transfected into each well using polyethyleneimine (Polysciences) in quadruplicate. Reporter expression was determined 48 hours after transfection by staining the cells with anti-NGFR-APC and anti-Thy1.1-eFluor450 (both eBiosciences) followed by staining with CYTOX Blue (Life Technologies) for live/dead cell discrimination. Events were acquired with a BD Fortessa. Data are expressed as the ratio between the median fluorescence intensity (MFI) values obtained for NGFR and the Thy1.1 MFI values followed by averaging of the quadruplicate measurements. The reporter expression values from samples transfected with the miR-142 expression vector are normalised to the values of the samples transfected with the empty expression vector, which was set to 1.

Western blot. Cells were washed in ice-cold PBS and lysed in RIPA buffer (Sigma), according to the manufacturer's instructions. The protein concentration was quantified using a Pierce BCA Protein Assay kit (Thermo Scientific) according to the manufacturer's instructions, and 20 μg protein used for each sample. The lysed samples were then boiled in laemmli buffer and proteins resolved using SDS-PAGE (Bio-rad) before being transferred to a nitrocellulose membrane. Blots were blocked with either 5% milk in Tris-buffered saline, 0.1% Tween-20 (TBST) or 5% Bovine Serum Albumin in TBS-Tween, and then probed with rabbit anti-mouse Pde3b antibody (SMCP3B) (NBPI-43333, Novus Biologicals). HRP-conjugated goat anti-rabbit IgG was used for secondary detection (GE healthcare) and polyclonal ß-actin antibody (4967, Cell Signalling Technology) used as an endogenous control. The blots were developed using enhanced chemiluminenscence (Thermo Scientific/Pearce), images acquired using image lab 6.0.1 software and the density of bands analysed using Fiji ImageJ software (72).

Statistical Analysis. All between-group differences in flow cytometric, RT-PCR, microarray and ELISA parameters were analysed using one-way ANOVA with Tukey's test. Two-tailed Student's t tests were used to analyse the flow cytometry based reporter assay system results. Statistical analyses were carried out using GraphPad Prism 6 (GraphPad Software). p<0.05 was considered statistically significant and represented in figures as (*), with p<0.01 represented in figures as (**), and p<0.001 represented as (***). Data represent mean±SEM.

Study Approval. All experiments were performed according to King's College London, London, U.K. and national guidelines, under a UK Home Office Project License (PPL:70/7869 to September 2018; P9720273E from September 2018).

### REFERENCES

1. Guo H, Ingolia N T, Weissman J S, Bartel D P. Mammalian microRNAs predominantly act to decrease target mRNA levels. Nature 2010; 466: 835-40.
2. Fabian M R & Sonenberg N. The mechanics of miRNA-mediated gene silencing: a look under the hood of miRISC. Nature Structural and Molecular Biology. 2012; 19:586-593.
3. O'Connell R M, Rao D S, Chaudhuri A A, Baltimore D. Physiological and pathological roles for microRNAs in the immune system. Nat. Rev. Immunol. 2010; 10: 111-122.
4. Pauley K, Cha S, Chan E K. MicroRNA in autoimmunity and autoimmune diseases. J. Autoimmun. 2009; 32: 189-194.
5. Lu J, et al. MicroRNA expression profiles classify human cancers. Nature. 2005; 435: 834-838.
6. Janssen H L, et al. Treatment of HCV Infection by Targeting MicroRNA. N. Engl. J. Med. 2013; 368: 1685-1694.
7. Brunkow M E, et al. Disruption of a new forkhead/winged-helix protein, scurfin, results in the fatal lymphoproliferative disorder of the scurfy mouse. Nat. Genet. 2001; 27: 68-73.
8. Godfrey V L, Wilkinson J E, Rinchik E M, Russell L B. Fatal lymphoreticular disease in the scurfy (sf) mouse requires T cells that mature in a sf thymic environment: potential model for thymic education. Proc Natl Acad Sci USA. 1991; 88:5528-32.
9. Godfrey V L, Wilkinson J E, Russell L B. X-linked lymphoreticular disease in the scurfy (sf) mutant mouse. Am J Pathol. 1991; 138:1379-87.
10. Powell B R, Buist N R M, Stenzel P. An X-linked syndrome of diarrhea, polyendocrinopathy, and fatal infection in infancy. The Journal of Pediatrics. 1982; 100(5):731-737.
11. Myers A K, Perroni L, Costigan C, Reardon W. Clinical and molecular findings in IPEX syndrome. Archives of Disease in Childhood. 2006; 91(1):63-64
12. Cobb B S, et al. A role for Dicer in immune regulation. J. Exp. Med. 2006; 203: 2519-2527.
13. Liston A, Lu L F, O'Carroll D, Tarakhovsky A, Rudensky A Y. Dicer-dependent microRNA pathway safeguards regulatory T cell function. J. Exp. Med. 2008; 205: 1993-2004.
14. Zhou X, et al. Selective miRNA disruption in TREGS cells leads to uncontrolled autoimmunity. J. Exp. Med. 2008; 205: 1983-1991.
15. Chong M M, Rasmussen J P, Rudensky A Y, Littman D R. The RNase III enzyme Drosha is critical in T cells for preventing lethal inflammatory disease. J. Exp. Med. 2008; 205: 2005-2017.
16. Landgraf P, et al. A mammalian microRNA expression atlas based on small RNA library sequencing. Cell. 2007; 129: 1401-1414.
17. Kuchen S, et al. Regulation of microRNA expression and abundance during lymphopoiesis. Immunity. 2010; 32: 828-39.
18. miRBase release 22. microRNA annotation and deep-sequencing data web site http://www.mirbase.org. Updated March 2018. Accessed Nov. 6, 2018.
19. Ding S, et al. Decreased microRNA-142-3p/5p expression causes CD4+ T cell activation and B cell hyperstimulation in systemic lupus erythematosus. Arthritis Rheum. 2012; 64:2953-63.
20. Talebi F, et al., MicroRNA-142 regulates inflammation and T cell differentiation in an animal model of multiple sclerosis. J. Neuroinflammation. 2017. 14:55.
21. Shakur Y, et al. Regulation and function of the cyclic nucleotide phosphodiesterase (PDE3) gene family. Prog. Nucleic Acid Res. Mol. Biol. 2001; 66:241-277.
22. Bopp T, et al. Cyclic adenosine monophosphate is a key component of regulatory T cell-mediated suppression. J. Exp. Med. 2007; 204:1301-1310.
23. Gavin M A, et al. FoxP3-dependent programme of regulatory T-cell differentiation. Nature. 2007; 445:771-775.
24. Whyte W A, et al. Master transcription factors and mediator establish super-enhancers at key cell identity genes. Cell. 2013; 153: 307-319.
25. Vahedi G, et al., Super-enhancers delineate disease-associated regulatory nodes in T cells. Nature. 2015; 520:558-562.
26. Hertweck A, et al. T-bet activates Th1 genes through mediator and the super elongation complex. Cell Rep. 2016; 15:2756-2770.
27. Rubtsov Y P, et al. Regulatory T Cell-derived Interleukin-10 limits inflammation at environmental interfaces. Immunity. 2008; 28:546-558.
28. Kramer N J, et al. Altered lymphopoiesis and immunodeficiency in miR-142 null mice.

Blood. 2015; 125(24):3720-30.
29. Loeb G B, et al. Transcriptome-wide miR-155 Binding Map Reveals Widespread Noncanonical MicroRNA Targeting. Mol Cell. 2012; 48(5):760-770.
30. Yu A, Zhu L, Altman N H, Malek T R. A Low Interleukin-2 Receptor Signaling Threshold Supports the Development and Homeostasis of T Regulatory Cells. Immunity. 2009; 30:204-217.
31. Bhairavabhotla R, et al. Transciptome profiling of human FoxP3+regulatory T cells. Hum Immunol. 2016; 77(2):201-213.
32. Singh Y, Garden O A, Lang F and Cobb B. MicroRNA-15b/16 enhances the induction of regulatory T cells by regulating the expression of rictor and mTOR. J. Immunol. 2015; 195(12):5667-77.
33. Warth S C, et al. Induced miR-99a expression represses Mtor cooperatively with miR-150 to promote regulatory T-cell differentiation. Embo J. 2015; 34(9):1195-1213.
34. Qin A, et al. MicroRNA-126 regulates the induction and function of CD4+ Foxp3+ regulatory T cells through PI3K/AKT pathway. Journal of Cellular and Molecular Medicine, 2013; 17(2):252-264.
35. Hippen K L, Loschi M, Nicholls J, MacDonald KPA, Blazar B R. Effects of microRNA on regulatory therapy to ameliorate graft-versus-host disease. Front Immunol. 2018; 9:57.
36. Zhang M, Zhang Q, Liu F, Yin L, Yu B, Wu J. MicroRNA-155 may affect allograft survival by regulating the expression of suppressor of cytokine signaling 1. Medical Hypotheses. 2011; 77(4):682-684.
37. Jiang S et al. Molecular dissection of the miR-17-92 cluster's critical dual roles in promoting Th1 responses and preventing inducible Treg differentiation. Blood, 2011; 118(20):5487-5497.
38. Negi, V, et al. Altered expression and editing of miRNA-100 regulates iTreg differentiation. Nucleic Acids Research. 2015; 43(16):8057-8065.
39. Takahashi H, et al. TGF-8 and retinoic acid induce the microRNA miR-10a, which targets Bcl-6 and constrains the plasticity of helper T cells. Nat Immunol. 2012; 13:587-595.
40. Liu, X et al. FOXP3 is a direct target of miR15a/16 in umbilical cord blood regulatory T cells. Bone Marrow Transplantation, 2014; 49(6):793-799.
41. Fayyad-Kazan, H, et al. MicroRNA profile of circulating CD4-positive regulatory T cells in human adults and impact of differentially expressed microRNAs on expression of two genes essential to their function. Journal of Biological Chemistry. 2012; 287(13):9910-9922.
42. Rouas R, et al. Human natural Treg microRNA signature: Role of microRNA-31 and microRNA-21 in FOXP3 expression. European Journal of Immunology. 2009; 39(6):1608-1618.
43. Pan W, et al. miR-124a targets effector programs to stabilize Treg-mediated immune homeostasis. Nat Commun. 2015; 12(6):7096.
44. Lu L F, et al. Function of miR-146a in controlling Treg cell-mediated regulation of Th1 responses. Cell. 2010; 142(6):914-29.
45. Huang B, et al. miR-142-3p restricts cAMP production in CD4+CD25− T cells and CD4+CD25+ TREG cells by targeting AC9 mRNA. EMBO Reports. 2009; 10(2):180-185.
46. Zheng Y, Josefowicz S Z, Kas A, Chu T T, Gavin M A, & Rudensky A Y. Genome-wide analysis of Foxp3 target genes in developing and mature regulatory T cells. Nature. 2007; 445(7130): 936-940.
47. Ebert M S & Sharp P A, Roles for microRNAs in conferring robustness to biological processes. Cell. 2012; 149:515-524.
48. Lerner A, Jeremias P, Matthias T. The World Incidence and Prevalence of Autoimmune Diseases is Increasing. International Journal of Celiac Disease. 2015; 3(4):151-155.
49. Maurice D H, Ke H, Ahmad F, Wang Y, Chung J, Manganiello V. Advances in targeting cyclic nucleotide phosphodiesterases. Nat. Rev. Drug Discovery. 2014; 13:290-314.
50. Kalop N, Chattipakorm S, Chattipakorn N. Effects of cilostazol in the heart. J. Cardiovasc. Med. 2011; 12:88-95.
51. Zou W. Regulatory T cells, tumour immunity and immunotherapy. Nature Reviews Immunology, 2006; 6(4):295-307.
52. Woo E Y et al. Regulatory CD4+CD25+ T cells in tumors from patients with early-stage non-small cell lung cancer and late-stage ovarian cancer. Cancer Research. 2001; 61(12):4766-4772.
53. Bates G J et al. Quantification of regulatory T cells enables the identification of high-risk breast cancer patients and those at risk of late relapse. Journal of Clinical Oncology. 2006; 24(34):5373-5380.
54. Curiel T J, et al. Specific recruitment of regulatory T cells in ovarian carcinoma fosters immune privilege and predicts reduced survival. Nature Medicine, 2004; 10(9):942-949.
55. Sasada T, Kimura M, Yoshida Y, Kanai M, Takabayashi A. CD4+CD25+regulatory T cells in patients with gastrointestinal malignancies: Possible involvement of regulatory T cells in disease progression. Cancer. 2003; 98(5):1089-1099.
56. Sato E et al. Intraepithelial CD8, 1-6. Website. papers2://publication/uuid/8C78742B-910F-4D67-9FAD-85CA30CD9EE3. Published 2005. Accessed Nov. 9, 2018.
57. Chen W, et al. Conversion of Peripheral CD4+CD25− Naive T Cells to CD4+CD25+ Regulatory T Cells by TGF-β Induction of Transcription Factor Foxp3. The Journal of Experimental Medicine, 2003; 198(12):1875-1886.
58. Seo N, Hayakawa S, Takigawa M, Tokura Y. Interleukin-10 expressed at early tumour sites induces subsequent generation of CD4+T-regulatory cells and systemic collapse of antitumour immunity. Immunology, 2001; 103(4):449-457.
59. Zarek P E, et al. A2Areceptor signaling promotes peripheral tolerance by inducing T-cell anergy and the generation of adaptive regulatory T cells. Blood, 2008; 111(1):251-259.
60. Juneja V R, et al. PD-L1 on tumor cells is sufficient for immune evasion in immunogenic tumors and inhibits CD8 T cell cytotoxicity. The Journal of Experimental Medicine, 2017; 214(4):895-904.
63. Choi Y H, et al. Alterations in regulation of energy homeostasis in cyclic nucleotide phosphodiesterase 3B-null mice. J. Clin. Invest. 2006; 116(12):3240-3251.
64. Huter E N, et al. TGFO-induced FoxP3+ Regulatory T Cells Rescue Scurfy Mice. Eur. J. Immunol 2009; 38(7):1814-1821.
65. B. D. Kubiak D, et al. Peritoneal negative pressure therapy prevents multiple organ injury in a chronic porcine sepsis and ischemia/reperfusion model. Shock 2010; 34(5):525-34.
66. Kanhere A, et al. T-bet and GATA3 orchestrate Th1 and Th2 differentiation through lineage-specific targeting of distal regulatory elements. Nat. Commun. 2012; 3:1268.
67. Samstein R M, et al. FoxP3 exploits a pre-existent enhancer landscape for regulatory T cell lineage specification. Cell 2012; 151:153-66.
68. Loven J, et al. Selective inhibition of tumor oncogenes by disruption of super-enhancers. Cell. 2013; 153:320-334.
69. Irizarry R A, et al. Exploration, normalization, and summaries of high density oligonucleotide array probe level data. Biostatistics. 2003; 4:249-64.
70. Ritchie M E, et al. limma power differential expression analyses for RNA-sequencing and microarray studies. Nucleic Acids Res. 2015; 43:e47.
71. Dabrowska M J, et al. Loss of MicroRNA targets in the 3′ untranslated region as a mechanism of retroviral insertional activation of growth factor independence. J. Virol. 2009; 83:8051-61.
72. Schindelin J, et al. Fiji: an open-source platform for biological-image analysis. Nature methods. 2012; 9(7):676-682.

### Tables

TABLE 1
miR-142 is the only microRNA associated with the highest ranked FOXP3-bound
super-enhancers in mouse T_{REGS}

| Enhancer rank | Chr | Start | Stop | Closest gene | Distance to closest TSS |
|---|---|---|---|---|---|
| 1 | chr8 | 129371463 | 129391915 | Tomm20 | 77906 |
| 2 | chr4 | 32337202 | 32345516 | Bach2 | −158893 |
| 3 | chr14 | 54846952 | 54853334 | Dad1 | 20270 |
| 4 | chr6 | 108486925 | 108503880 | Itpr1 | within gene |
| 5 | chr11 | 87558486 | 87568424 | Mir142 | −1941 |
| 6 | chr6 | 129179073 | 129184784 | 2310001H17Rik | within gene |
| 7 | chr15 | 61996321 | 62001568 | Pvt1 | within gene |
| 8 | chr19 | 53495621 | 53499465 | Smndc1 | −30557 |
| 9 | chr12 | 102121473 | 102133871 | Gpr68 | within gene |
| 10 | chr10 | 13702612 | 13713611 | Hivep2 | within gene |
| 11 | chr16 | 49790990 | 49799225 | Cd47 | −56541 |
| 12 | chr17 | 84729938 | 84732636 | Thada | within gene |
| 13 | chr17 | 29530547 | 29532572 | Fgd2 | 32688 |
| 14 | chr15 | 72980095 | 72992645 | Eif2c2 | within gene |
| 15 | chr17 | 52301754 | 52302516 | Gm20098 | 279702 |
| 16 | chr13 | 43870595 | 43873683 | Cd83 | −6792 |
| 17 | chr10 | 95833510 | 95837468 | Btg1 | −242166 |
| 18 | chr12 | 74694414 | 74706077 | Prkch | within gene |
| 19 | chr11 | 34495132 | 34495979 | Dock2 | within gene |
| 20 | chr19 | 53515926 | 53517976 | 5830416P10Rik | within gene |

TABLE 2
Conservation of miR-142-5p target sequences
across species in the Pde3b 3′UTR.

| | |
|---|---|
| Pde3b 3′UTR 1550: | |
| Bos taurus (cow): | 5′ UUUAAUGAAUCACUAAGCUUUAUU 3′ |
| | (SEQ ID NO: 5) |
| Sus scrofa (pig): | 5′ UUUAAUGAAUUACUAAGCUUUAUU 3′ |
| | (SEQ ID NO: 6) |
| Felis Catus (cat): | 5′ UUUAAUGAAUCACCGAGCUUUAUU 3′ |
| | (SEQ ID NO: 7) |
| Rattus norvegicus | 5′ UUUAAUGAAUCACUACACUUUAUU 3′ |
| (rat): | (SEQ ID NO: 8) |
| Pan troglodytes | 5′ UUUAAUGAAUCACUAAGCUUUAUU 3′ |
| (chimp): | (SEQ ID NO: 9) |
| Homo sapiens (human): | 5′ UUUAAUGAAUCACUAAGCUUUAUU 3′ |
| | (SEQ ID NO: 4) |
| Mus musculus (mouse): | 5′ UUUAAUGAAUCACUACACUUUAUU 3′ |
| (SEQ ID NO: 10) | \|\| \|\| \| \|\| \|\|\|\|\|\| |
| miR-142-5p: | 3′ UCAUCAC-GAAAGA--UGAAAUAC 5′ |
| (SEQ ID NO: 1) | |

1. A T regulatory cell (Treg) in which the level of microRNA miR-142-5p (CAUAAAGUAGAAAGCACUACU) or a variant thereof is increased or decreased.

2. The Treg of claim 1, wherein said variant comprises or consists of a nucleotide sequence with a sequence identity of at least 70% to said miR-142-5p sequence, or wherein said variant comprises or consists of a nucleotide sequence containing up to 8 altered nucleotides in said miR-142-5p sequence.

3. The Treg of claim 1 or claim 2, wherein said variant comprises or consists of a nucleotide sequence in which all of the nucleotides corresponding to nucleotides 2 to 7 of said miR-142-5p sequence are retained.

4. The Treg of any one of claims 1 to 3, wherein said Treg is a recombinant Treg.

5. The Treg of any one of claims 1 to 4, wherein said Treg is genetically modified or genetically engineered.

6. The Treg of any one of claims 1 to 5, wherein a polynucleotide encoding said miR-142-5p or a variant thereof is integrated into the genome of the Treg cell to increase the level of miR-142-5p (CAUAAAGUAGAAAGCACUACU) or a variant thereof.

7. The Treg of any one of claims 1 to 6, wherein additional copies of a polynucleotide encoding miR-142-5p or a variant thereof are inserted into said Treg cell to increase the level of miR-142-5p (CAUAAAGUAGAAAGCACUACU) or a variant thereof.

8. The Treg of any one of claims 1 to 5, wherein the endogenous genomic sequence encoding miR-142-5p is deleted or mutated in said Treg cell to decrease the level of miR-142-5p (CAUAAAGUAGAAAGCACUACU) or a variant thereof.

9. The Treg of any one of claims 1 to 8, wherein said Treg is a human Treg.

10. The Treg of any one of claims 1 to 9, for use in therapy.

11. The Treg of claim 10, wherein the level of miR-142-5p (CAUAAAGUAGAAAGCACUACU) or a variant thereof is increased, for use in the treatment of autoimmune disease.

12. The Treg of claim 10, wherein the level of miR-142-5p (CAUAAAGUAGAAAGCACUACU) or a variant thereof is decreased, for use in the treatment of cancer.

13. The Treg for use as claimed in any one of claims 10 to 12, wherein said therapy or treatment is an autologous therapy.

14. A method for preparing Tregs suitable for use in the treatment of autoimmune disease, said method comprising the following steps:
i) Isolating Tregs from a sample taken from a subject, preferably a blood sample;
ii) Modifying the Tregs so that the level of miR-142-5p (CAUAAAGUAGAAAGCACUACU) or a variant thereof is increased; and optionally
iii) in vitro expansion of the Treg cells.

15. A method for preparing Tregs suitable for use in the treatment of cancer, said method comprising the following steps:
i) Isolating Tregs from a sample taken from a subject, preferably a blood sample;
ii) Modifying the Tregs so that the level of miR-142-5p (CAUAAAGUAGAAAGCACUACU) or a variant thereof is decreased; and optionally
iii) in vitro expansion of the Treg cells.

16. The method of claim 14 or claim 15, wherein said variant is as defined in any one of claims 2 to 3 or said Treg is as defined in any one of claims 4 to 9.

17. A population of Tregs obtainable by the method of any one of claims 14 to 16.

18. An agent which inhibits or reduces phosphodiesterase-3b (PDE3B) levels or activity, for use in the treatment of autoimmune disease.

19. The agent for use of claim 18, wherein said agent is selective for inhibition of PDE3B over the inhibition of PDE3A.

20. The agent for use of claim 18 or claim 19, wherein said agent is miR-142-5p (CAUAAAGUAGAAAGCACUACU) or a variant thereof.

21. The agent for use of any one of claims 18 to 20, wherein said inhibition or reduction in PDE3B levels or activity is in T regulatory cells (Tregs).

22. A method of reducing PDE3B levels in a cell, wherein said method comprises the use of miR-142-5p (CAUAAAGUAGAAAGCACUACU) or a variant thereof.

23. The agent for use of claim 20 or claim 21, or the method of claim 22, wherein said variant is as defined in any one of claims 2 to 3.

24. A method of treating autoimmune disease in a subject, said method comprising the step of administrating an effective amount of a regulatory T cell (Treg) of any one of claims 1 to 9 in which the level of miR-142-5p (CAUAAAGUAGAAAGCACUACU) or a variant thereof is increased, to said subject.

25. A method of treating cancer in a subject, said method comprising the step of administrating an effective amount of a regulatory T cell (Treg) of any one of claims 1 to 9 in which the level of miR-142-5p (CAUAAAGUAGAAAGCACUACU) or a variant thereof is decreased, to said subject.

26. The use of a regulatory T cell (Treg) of any one of claims 1 to 9 in which the level of miR-142-5p (CAUAAAGUAGAAAGCACUACU) or a variant thereof is increased, in the manufacture of a medicament, or composition, for the treatment of autoimmune disease.

27. The use of a regulatory T cell (Treg) of any one of claims 1 to 9 in which the level of miR-142-5p (CAUAAAGUAGAAAGCACUACU) or a variant thereof is decreased, in the manufacture of a medicament, or composition, for the treatment of cancer.

28. A method of treating autoimmune disease in a subject, said method comprising the step of administrating an effective amount of an agent which inhibits or reduces PDE3B levels or activity, to said subject.

29. The use of an agent which inhibits or reduces PDE3B levels or activity, in the manufacture of a medicament, or composition, for the treatment of autoimmune disease.

30. The method or use of claim 28 or 29, wherein said agent is as defined in any one of claims 19 to 21 or claim 23.
